(19) United States
(12) Patent Application Publication
(10) Pub. No.: US 2013/0274838 A1
(43) Pub. Date: Oct. 17, 2013

(54) OPTICAL CONTROL OF CARDIAC FUNCTION

(71) Applicants: Emilia Entcheva, Stony Brook, NY (US); Zhiheng Jia, East Setauket, NY (US); Zhongju Lu, East Setauket, NY (US); Harold Bien, Stony Brook, NY (US); Ira S. Cohen, Stony Brook, NY (US)

(72) Inventors: Emilia Entcheva, Stony Brook, NY (US); Zhiheng Jia, East Setauket, NY (US); Zhongju Lu, East Setauket, NY (US); Harold Bien, Stony Brook, NY (US); Ira S. Cohen, Stony Brook, NY (US)

(73) Assignee: The Research Foundation of State University of New York, Albany, NY (US)

(21) Appl. No.: 13/880,236

(22) PCT Filed: Oct. 18, 2011
(86) PCT No.: PCT/US11/56716

§ 371 Date: Jul. 1, 2013

Related U.S. Application Data
(60) Provisional application No. 61/394,256, filed on Oct. 18, 2010.

(51) Int. Cl. A61N 5/06

(52) U.S. Cl. CPC A61N 5/0622
USPC 607/89; 607/88

(57) ABSTRACT

The invention features an optically-controlled biological device that includes a biological component comprising a non-excitable cell expressing a light-gated ion channel protein and capable of forming gap junction channels with a target cell, and an optical stimulation unit.

### CROSS REFERENCE TO RELATED APPLICATIONS

This application claims priority to U.S. Application No. 61/394,256 filed Oct. 18, 2010, which is incorporated herein by reference in its entirety.

### FIELD OF THE INVENTION

The invention features an optically-controlled biological device comprising a non-excitable cell expressing a light-gated ion channel protein and capable of forming gap junction channels with cardiomyocytes, and an optical stimulation unit.

### BACKGROUND OF THE INVENTION

The heart's natural pacemaker is a small mass of specialized cells called the sinoatrial (SA) node, which initiates and maintains the heart's normal rhythm (referred to as normal sinus rhythm). The sinoatrial node consists of only a few thousand electrically active pacemaker cells that generate spontaneous rhythmic action potentials that subsequently propagate to induce coordinated muscle contractions of the atria and ventricles. The rhythm is modulated, but not initiated, by the autonomic nervous system.

Thus, normal cardiac depolarization is initiated by the SA node. From the sinus node the wave of depolarization spreads across the atria to the atrioventricular (AV) node. At the AV node the impulse is delayed briefly and atrial contraction is completed. The depolarization wave then proceeds to the bundle of His where it follows two pathways, travelling along the right and left bundle branches. The impulse travels the length of the bundles along the interventricular septum to the base of the heart. The bundles divide into the Purkinje system, which distributes the wave of depolarization to the ventricle walls, initiating ventricular contraction.

Cardiac disease can result, for example, when an event disrupts or alters the generation of the impulse or disrupts or alters the conduction of the impulse to the atria or ventricles. For example, when an event interrupts the heart's normal beat, either intermittent or sustained arrhythmias can occur. Cardiac arrhythmia is a group of conditions in which the muscle contraction of the heart is irregular and/or is faster (tachycardia) or slower (bradycardia) than normal. The most common causes of bradycardias are reduced SA node pacemaker activity and depressed conduction. Slowing of the SA node pacemaker, sinus bradycardia, can be caused by excessive parasympathetic tone, hyperthyroidism, and administration of drugs including β-adrenergic blockers and calcium channel blockers. In addition, sinus bradycardia occurs in sinus node dysfunction (tachycardia-bradycardia syndrome) commonly seen in the elderly. Depressed conduction causing bradycardia can occur, for example, when conduction of the sinus impulse to the ventricles is impaired (referred to as a block). Three regions of the heart are particularly vulnerable to block, SA node, the AV node, and the His-Purkinje system.

Thus, SA block occurs when impulses arising in the SA node fail to depolarize the atria. AV block is clinically sorted into three categories: first-degree AV block is an abnormal delay in the AV conduction (prolonging the PR interval); second-degree AV block is more severe because some, but not all, P waves fail to activate the ventricles; and third-degree AV block no impulses are conducted from the atria to the ventricles. Second-degree AV block may in turn be categorized as type 1, in which the blocked P waves are proceeded by progressive prolongation of the PR interval; and type 2, in which there is no such pattern. Type 2 second-degree AV block is often a result of lesions of the His-Purkinje system.

A tachyarrythmia is a tachycardia associated with an irregularity in the normal heart rhythm. Tachyarrythmias include tachycardia (a series of premature systoles), fibrillation (disorganized activation where there is no effective beating), and flutter (a rapid regular activation of the atria or ventricle). Mechanisms that account for tachyarrhythmias include accelerated pacemaker activity, reentry, and triggered depolarizations. Accelerated firing of a pacemaker cell of the SA node causes sinus tachycardia. Reentry occurs when a single impulse traveling through the heart gives rise to two or more propagated responses. Reentrant arrhythmias may be caused by abnormal conduction (decremental conduction and unidirectional block), inhomogeneties of the action potential, and abnormal conducting structures.

Malfunction or loss of pacemaker cells can occur due to disease or aging. For example, acute myocardial infarction kills millions of people each year and generally induces in survivors marked reductions in myocyte number and cardiac pump function. Adult cardiac myocytes divide only rarely, and the usual responses to myocyte cell loss include compensatory hypertrophy and/or congestive heart failure, a disease with a significant annual mortality.

A variety of implantable devices have been developed to address cardiac pacing disorders. Electronic pacemakers are lifesaving devices that provide a regular heartbeat in settings where the sinoatrial node, atrioventricular conduction, or both, have failed. They also have been adapted to the therapy of congestive heart failure. One of the major indications for electronic pacemaker therapy is high degree of heart block, such that a normally functioning sinus node impulse cannot propagate to the ventricle, resulting in ventricular arrest and/or fibrillation, and death. Another major indication for electronic pacemaker therapy is sinoatrial node dysfunction, in which the sinus node fails to initiate a normal heartbeat, thereby compromising cardiac output.

Implantable cardioverter defibrillators (ICDs) are used to treat patients at risk for recurrent, sustained ventricular tachycardia or fibrillation. These devices deliver electrical pulses or shocks to help correct the irregular heartbeats.

### SUMMARY OF THE INVENTION

In a first aspect, the invention features an optically-controlled biological device comprising: (a) a non-excitable cell expressing a light-gated ion channel protein and capable of forming a gap junction channel with a cardiomyocyte when implanted in a subject, and (b) an optical stimulation unit. In one embodiment the device is a cardiac pacemaker. In one embodiment, the device is an implantable cardioverter defibrillator. In one embodiment, the device is an optical-mechanical actuator.

In another aspect, the invention features a method of treating a subject with a cardiac pacing disorder, comprising: (a) delivering in proximity to the heart of the subject a non-excitable cell expressing a light-gated ion channel protein, wherein the non-excitable cell forms a gap junction channel with a cardiomyocyte of the subject; and (b) providing an optical stimulation unit.

In one embodiment, the light-gated ion channel protein is one or more proteins selected from the group consisting of channelrhodopsin-1 (ChR1), channelrhodopsin-2 (ChR2), Volvox channelrhodopsin (VChR1), halorhodopsin (Halo/NpHR), archaerhodopsin-3 (Arch), and Leptosphaeria maculans rhodopsin (Mac).

In one embodiment, the non-excitable cell is engineered to express one or more connexin proteins. In one embodiment, the non-excitable cell is engineered to express one or more connexins selected from the group consisting of connexin 40, connexin 43, and connexin 45. In one embodiment, the non-excitable cell endogenously expresses one or more connexin proteins.

In one embodiment, the non-excitable cell is a stem cell, an endothelial cell, a fibroblast or an adipocyte. In one embodiment, the non-excitable cell is a stem cell. In other embodiments the non-excitable cell is pluripotent or totipotent. In other embodiments the non-excitable cell is an embryonic stem cell, an induced pluripotent stem cell, or a mesenchymal stem cell. In one embodiment, the non-excitable cell is a human mesenchymal stem cell. In one embodiment the non-excitable cell is substantially incapable of differentiating after it is implanted in the subject.

In one embodiment, the optically-controlled biological device comprises between about 5,000 to about 1.5 million cells. In other embodiments the biological device comprises between about 700,000 to about 1.0 million cells. In one embodiment, the biological device comprises at least about 5,000 cells; at least about 10,000 cells; at least about 50,000 cells; at least about 100,000 cells; at least about 200,000 cells; at least about 500,000 cells; at least about 700,000 cells; or at least about 1 million cells.

In one embodiment, the optically-controlled biological device comprises a number of non-excitable cells such that the ratio of non-excitable cells to target cells is from about 1:20 to about 1:100; from about 1:20 to about 1:500; from about 1:30 to about 1:70; from about 1:50 to about 1:100; from about 1:100 to about 1:200; from about 1:200 to about 1:300; from about 1:300 to about 1:400; from about 1:400 to about 1:500; and from about 1:500 to about 1:1000.

In one embodiment, the optical stimulation unit comprises a light source wherein the light source a laser, a laser diode, a light emitting diode, an organic light emitting diode, an incandescent light source, or an organic light source. In one embodiment, the optical stimulation unit comprises one or more sensors for detecting cardiac pacing in the atrium, ventricles, or both.

In one embodiment, the cardiac pacing disorder is selected from the group consisting of cardiac arrhythmia, reentrant arrhythmia, bradycardia, tachycardia, sinus bradycardia, sinus tachycardia, ventricular tachycardia, supraventricular tachycardia, ventricular fibrillation, atrial flutter, atrial fibrillation, pro-arrhythmic cardiac alternans, sinus node dysfunction, first degree heart block, type 1 second degree heart block, type 2 second degree heart block, third degree heart block, SA block, and SV block.

In one embodiment, the non-excitable cell is delivered to one or more of the sinoatrial node, the atrioventricular node, Bachmann's bundle, the atrioventricular junction region, His branch, left bundle branch, right bundle branch, Purkinje fibers, right atrial muscle, left atrial muscle, or ventricular muscle.

### DESCRIPTION OF THE FIGURES

FIG. 1. Illustration of the functional “tandem cell unit” concept of donor-host cells. Non-excitable cells (HEK cells, in this case) are transfected to express a light-sensitive ion channel (ChR2). When coupled via gap junctions to excitable cardiomyocytes (CM) they form a functional “tandem cell unit” that is optically controllable, i.e., the CM will generate an action potential upon light-triggered opening of the depolarizing ChR2 in the HEK cell.

FIG. 2. Development and functional characterization of a cell delivery system for ChR2. a, b Expression of ChR2 in HEK cells via EYFP reporter in 1^{st} and 10^{th} passage after transfection and purification; scale bar is 50 μm. c: Voltage-clamp records in the developed HEK cell line under ramp protocol and optical excitation (see inset) show a robust inward/depolarizing current. Red and blue traces are with light off at different potentials, while green and black traces are with light on (470 nm). d: Magnitude of the light-triggered current does not depend on the duration of rest (Δt rest) or activation (Δt act), thus indicating relatively fast recovery from inactivation in the examined range. Holding potential is −80 mV. e: Kinetics of activation (on) and deactivation (off), quantified by a τₛₗ parameter in the sigmoid curve fits to the light-controlled current transitions (see inset) bar graphs represent mean±SEM.

FIG. 3. Development and functional characterization of a cell delivery system for ChR2. a: Voltage-clamp test protocol and example traces for quantification of the steady-state ChR2 current in single HEK-ChR2 cells with 500 ms voltage pulses in the range (−80 to +50 mV) with and without excitation light for ChR2 on (470 nm, 0.24 mW/mm²). b: Example curves for the light-sensitive component after subtraction of current in dark, and the resultant average current-voltage (l−V) relationship for n=12 cells, cell capacitance 43.3±7.5 pF, data are presented as mean±SD.

FIG. 4. Implementation of the “tandem cell unit” concept via co-culture of CM and HEK+ChR2. a: Phase and fluorescence (EYFP) images of CM+HEK+ChR2 co-culture. Scale bar is 30 μm. b: Phase and immunocytochemistry images of CM+HEK+ChR2 co-culture demonstrating connectivity between the two cell types via gap junctions. Nuclei are labeled by DAPI (blue); α-sarcomeric-actinin (CM-specific) is in red and Cx43 in green. Scale bar is 10 μm.

FIG. 5. Implementation and validation of the TCU concept for neonatal rat CM and adult canine CM coupled to HEK+ChR2 cells. a: Phase and fluorescence images of neonatal rat CM and HEK-ChR2 co-culture. Immunostaining in red for α-actinin (CMs), green is EYFP-ChR2-expressing HEK cells, typically forming small clusters as shown. Scale bar is 20 μm. b: Western blot for Cx43 and α-tubulin (at 55 kD) in the cell delivery system (HEK-ChR2), column 2; column 1 shows a positive control of stably transfected HeLa-Cx43 cells; column 3 shows parental HEK cells without ChR2; column 4 shows the ladder—MagicMark™ bands in kDa; Normalized (Cx43/tubulin) expression is provided for four gels (mean±95% CI). c: Histogram of measured coupling conductances in TCUs of canine CMs and HEK-ChR2 cells, n=31, median value of 4 nS and IQR (2-11 nS); red arrow indicates coupling levels allowing optical excitability of the TCUs. d: Dual whole-cell voltage clamp of a TCU—adult canine ventricular CM (1) and HEK-ChR2 cell (2). Voltage steps (V1=10 mV, 0.4 s), applied to the canine CM (cell 1), induced junctional currents (I2) in this cell pair (estimated g.j. conductance of 11 nS). e: Action potentials in a cell pair (canine CM and HEK-ChR2 cell, phase image on the left) in response to optical pacing (0.13 mW/mm², 10 ms pulses). Due to coupling, the HEK cell exhibits a low-pass filtered version of the CM-generated action potentials. f: Action potentials in a cell pair (canine CM and HEK-ChR2 cell) in response to continuous optical pacing before, during and after washout of uncoupler carbenoxolone (CBX).

FIG. 6. Optical control of cardiac tissue function over space-time: light-triggered excitation waves and light-triggered contractions. a: Experimental setup for ultra-high resolution high-speed optical imaging and optical control of cardiac excitation. 1) experimental chamber with tangential light illumination for calcium imaging (Rhod4, 525 nm), focused LED illumination on a moveable stage at the bottom for ChR2 excitation (470 nm); emitted calcium-dye fluorescence is at 585 nm, see enlarged depiction to the right; 2) high-NA optics for high-resolution macroscopic imaging—50 mm f/1.0 Navitar lens and emission filter; FOV is 2.5 cm, resolution—about 22 μm/pix; 3) Gen III MCP intensifier; 4) pco 1200 hs CMOS camera 1200×1084 pix, 200 fps full frame, with 7 GB on board memory; 5) Light source, excitation filter and optical light guides for tangential excitation; 6) computer system and software for data acquisition and control of electrical and optical stimulation; 7) USB timer/counter interface for stimulation control; 8) controllable stimulator for electrical pacing (analog output); 9) controllable stimulator for optical nacing (TTL output). 10) Blue LED (470 nm) for ChR2 excitation, driven by the TTL stimulator output. b: Activation maps in a cardiac monolayer by electrical and optical stimulation. Color represents time of activation; isochrones are shown at 50 ms. Calcium transient traces are shown from 2 locations (A and B), normalized fluorescence. The red trace shows optical stimulation signal. c: Normalized Ca²⁺ transients from CM monolayer (solid black line) and CM+HEK+ChR2 co-culture (dashed blue line). d: Quantification of calcium transient duration (CTD) from n=3 cultures—shown are CTD25, CTD50 and CTD80 for CM monolayer and CM+HEK+ChR2 co-culture. Data are shown as mean±SE. e: Comparison of conduction velocity (CV) at room temperature in CM only (n=7), CM+HEK (n=6), CM+HEK+ChR2 with electrical pacing and optical pacing in the same samples (n=8). Data are shown as mean±95% CI. f: Example contractility recording from optically-driven CM+HEK+ChR2—displacement normalized to cell length. Scale bar is 1 s.

FIG. 7. Optical control of cardiac tissue function over space-time: light-triggered excitation waves and light-triggered contractions. Experimental setup as in FIG. 6a. a: Activation maps in a cardiac monolayer by electrical and optical pacing at 0.5 Hz. Color represents time of activation; isochrones are shown in black at 0.15 s. Calcium transient traces in response to electrical or optical stimulation are shown from 2 locations (A and B), normalized fluorescence. Horizontal marks indicate time of stimulation (electrical pulses were 10 ms, optical—20 ms each). b: Normalized Ca²⁺ transients from CM monolayer (red), CM:HEK (black) and CM:HEK+ChR2 co-culture 100:1 (blue) at 1 Hz pacing. c: Quantification of calcium transient duration (CTD)—CTD25, CTD50 and CTD80 for pure CM monolayer, 45:1 and 100:1 CM:HEK, as well as 100:1 CM:(HEK+ChR2) co-culture under electrical and optical pacing at 1 Hz. e: Comparison of conduction velocity (CV) among the same 5 groups as in (c); for c and d optical pacing was at irradiance of 0.01-0.04 mW/mm², 50 ms pulses; data are shown as mean±95% CI; listed number of samples applies to both; e: Strength-duration curve (along with the equation for the fitted curve) obtained for optical pacing in co-cultures (100:1 CM:HEK ratio) at 30° C., n=8, mean±SEM.

FIG. 8. Direct expression of light-sensitive channels in cardiac cells via electroporation. a: Cardiac myocytes express ChR2—EYFP at low efficiency after electroporation (left), control (right); scale bar is 50 μm. Panel b: Cardiac fibroblasts robustly express ChR2 after electroporation. Data in the bar graphs are mean±SE. c: Phase and fluorescence (EYFP) images of two electroporation-transfected cardiomyocytes, for which movies of optically-triggered contractions are provided. Scale bar is 20 μm.

FIG. 9. Direct expression of light-sensitive channels in mesechnymal stem cells via electroporation. Expression of ChR2 in mesenchymal stem cells—canine (cMSC) and human (hMSC). cMSC showed substantially better expression than hMSC. Scale bar is 20 μm. Data in the bar graphs are mean±SEM. Normalized fluorescence is the image's total fluorescence normalized by the mean fluorescence from control (non-transfected) cells of the same type.

FIG. 10. Equivalent circuit for TCU-mediated excitation of cardiac tissue. a: CM-CM cell pair where both cell carry the excitatory current, and equivalent circuit. b: TCU of a donor (D) cell and a CM, with differences listed. c: abstraction using a Source-Neighbor-Load (S-N-L) triad for a spatially-extended system. d: simplified equivalent circuit of the S-N-L; arrows indicate the direction of contribution of the different circuit elements towards “ease of excitation”, as analyzed in example 5.

### DETAILED DESCRIPTION

The invention features an optically-controlled biological device comprising (a) a biological component comprising a non-excitable cell expressing a light-gated ion channel protein and capable of forming a gap junction channel with a target cell (e.g., a cardiomyocyte), and (b) an optical stimulation unit.

### Non-Excitable Cell

In one aspect the invention features a donor cell engineered to express a light-gated ion channel protein, which forms a tandem cell unit with a target cell via gap junction channels between the donor and target cell. Stimulation of the donor cell by light causes depolarization or repolarization of the membrane potential of the donor cell, which in turn triggers depolarization or repolarization of the target cell. In one embodiment the donor cell is a non-excitable cell.

In one embodiment, a non-excitable cell that is capable of forming a gap junction with the target cell is engineered to express a light-gated ion channel. A non-excitable cell is a cell that does not normally generate an action potential and possesses only passive conduction properties. However, the non-excitable cell may be manipulated such that it will contribute to the depolarization and/or repolarization of an excitable cell to which it is coupled in response to certain stimuli, as in the present invention, when such a cell is engineered to express a light-gated ion channel. Cell types include, but are not limited to, stem cells, fibroblasts, endothelial cells, adipocytes, or other cell that can be implanted in a subject.

An implantable cell means a cell that can be implanted or administered into a subject. Preferably, the biological component of the present invention comprises an implantable cell capable of gap junction-mediated communication with cardiomyocytes or other target cells. In one embodiment, the cell is substantially incapable of differentiation when implanted into a subject. In other embodiments the cell is pluripotent or totipotent. In one embodiment, the cell is an embryonic or adult stem cell. In one embodiment, the cell is an induced pluripotent stem cell. In one embodiment, the cell is an adult mesenchymal stem cell. In one embodiment, the cell is an adult human mesenchymal stem cell.

The absence of rejection, if nonautologous cells are employed, will facilitate the long-term use of the cell-based biological component. In this regard, cells could be obtained from an autologous source. Thus, in one embodiment, the non-excitable cell is obtained from the subject and manipulated to express a light-activated ion channel. In a further embodiment the cell is engineered to express one or more connexins. In other embodiments, the non-excitable cell is immunoprivileged. For example, evidence suggests that hMSCs cells are immunoprivileged [1]. In addition, no cellular or humoral rejection was evident six weeks following injection of hMSCs into canine hearts [2]. Thus, allogeneic solutions based on the immunoprivileged status of hMSCs provides off-the-shelf cells, that could be ready for implantation.

### Light-Gated Ion Channels

Light-gated ion channels are transmembrane proteins that form an ion channel that opens and/or closes in response to light.

TABLE 1
Light-sensitive ion channels currently used in mammalian cells.

| Light-Sensitive | GenBank | | Exc. | | |
|---|---|---|---|---|---|
| Ion Channel | Accession No. | Current | wavelength | Notes | References |
| Channelrhodopsin- | AF385748 | depolarizing | 470 nm | high-intensity | [3-5] |
| 1 (ChR1) | | | | response | |
| Channelrhodopsin- | AF461397 | depolarizing | 470 nm | low-intensity | [5-10] |
| 2 (ChR2) | | | | response | |
| Volvox | EU285659 | depolarizing | 535 nm | | [11, 12] |
| Channelrhodopsin | | | | | |
| (VChR1) | | | | | |
| Halorhodopsin | EF474018 | repolarizing | 589 nm | | [13-18] |
| (Halo/NpHR) | | | | | |
| Archaerhodopsin-3 | D50848 | repolarizing | 575 nm | fast | [16, 19] |
| (Arch) | GU045593 | | | spontaneous | |
| | | | | recovery | |
| Leptosphaeria | AF290180 | repolarizing | 470 nm | | [16, 20] |
| maculans (Mac) | | | | | |

Light-gated ion channels useful in the present invention include, but are not limited to those listed in Table 1, as well as mutations and other variations and alterations of the light-gated ion channels such as chimeras and light-gated ion channels comprising one or more deletions or insertions. Such mutations or alterations may, for example, alter the wavelength of light at which the light-gated ion channel is activated, alter the specificity of the channel for certain ions, and/or alter the kinetics of the light-gated ion channel. For example, C128 mutations of ChR2 provide a light-gated ion channel that can be opened by a blue light pulse and closed by a green or yellow light pulse. Mutating the E123 residue, on the other hand, can accelerate channel kinetics. A C-terminal truncation of ChR2 (ChR21-315) is substantially as active as the full-length protein. Light-gated ion channels also include variations that have been mammalian codon-optimized.

A chimera of a light-gated ion channel comprises portions of more than one type of light-gated ion channels. For example, a chimera may comprise portions of ChR1 and ChR2 or VChR1, and so forth. In addition, a light-gated ion channel chimera includes an ion channel comprising portions of an light-gated ion channel derived from different species. For example, one portion of the channel may be derived from a human and another portion may be derived from a non-human.

### Connexins

For the tandem cell unit to be functional (to fire an action potential upon light excitation), proper gap junctional coupling between the donor and target cell is needed to close the ion current loop. The major connexins expressed in human cardiac muscle are Cx43, Cx40, and Cx45. Gap junction channels can be formed when the target and donor cells express one or more connexins.

The cDNA sequence and deduced amino acid sequence of connexin 40 (Cx40, also known as GJA5) has been characterized from several species including mouse, rat, and human. Sequences of the Cx40 proteins are available from public databases. The GenBank accession number for human Cx40 is NM_{—}181703.

The cDNA sequence and deduced amino acid sequence of connexin 43 (Cx43, also known as GJA1) has been characterized from several species including mouse, rat, and human. Sequences of the Cx43 proteins are available from public databases. The GenBank accession number for human Cx43 is BC026329.

The cDNA sequence and deduced amino acid sequence of connexin 45 (Cx45, also known as GJC1) has been characterized from species including mouse and human. Sequences of the Cx45 proteins are available from public databases. The GenBank accession number for human Cx45 is NM_{—}005497.

### Sequence Similarity and Identity

The use of a particular light-gated ion channel isoform as described herein (e.g., the expression of the light-gated ion channel in a non-excitable cell) encompasses the use of a light-gated ion channel exhibiting at least about 75%, at least about 80%, at least about 85%, at least about 90%, or at least about 95% identity with that isoform. In embodiments of the invention comprising portions of a particular light-gated ion channel isoform, for example, the use of a N-terminal portion of a particular isoform encompasses the use of a N-terminal portion of the channel exhibiting at least about 60%, at least about 70%, at least about 80%, or at least about 90% identity with the N-terminus of that isoform. In addition, the use of a C-terminal portion of a particular light-gated ion channel isoform encompasses the use of a C-terminal portion of a light-gated ion channel exhibiting at least about 60%, at least about 70%, at least about 80%, or at least about 90% identity with the C-terminus of that isoform.

The use of a particular connexin isoform as described herein (e.g., the expression of the connexin in a non-excitable cell) encompasses the use of a connexin exhibiting at least about 75%, at least about 80%, at least about 85%, at least about 90%, or at least about 95% identity with that isoform. In embodiments of the invention comprising portions of a particular connexin isoform, the use of a N-terminal portion of a particular connexin isoform encompasses the use of a N-terminal portion of a connexin exhibiting at least about 60%, at least about 70%, at least about 80%, or at least about 90% identity with the N-terminus of that connexin isoform. In addition, the use of a C-terminal portion of a particular connexin isoform encompasses the use of a C-terminal portion of a connexin exhibiting at least about 60%, at least about 70%, at least about 80%, or at least about 90% identity with the C-terminus of that connexin isoform.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, at least 40%, at least 50%, at least 60%, or at least 70%, 80%, or 90% or more of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The percent identity between two light-gated ion channel amino acid sequences or between two connexins amino acid sequences can be determined using the Needleman et al. algorithm [28]. Another non-limiting example of a mathematical algorithm utilized for the comparison of sequences is the algorithm of Myers and Miller, CABIOS (1989).

The invention also encompasses polypeptides having sufficient similarity so as to perform one or more of the same functions performed by the light-gated ion channel or the connexin molecule. Thus, the light-gated ion channel or connexin includes isoforms exhibiting at least about 75%, at least about 80%, at least about 85%, at least about 90%, or at least about 95% similarity with the light-gated ion channel or connexin protein. Similarity is determined by considering conserved amino acid substitutions. Such substitutions are those that substitute a given amino acid in a polypeptide by another amino acid of like characteristics. Conservative substitutions are likely to be phenotypically silent. Guidance concerning which amino acid changes are likely to be phenotypically silent are found in the art (see e.g., [21]). The comparison of sequences and determination of percent identity and similarity between two polypeptides can be accomplished using mathematical algorithms known in the art. (see e.g., [22-26]). A non-limiting example of such a mathematical algorithm is described in Karlin et al. [27].

A light-gated ion channel or connexin according to the present invention, may also be a polypeptide encoded by a nucleic acid sequence capable of hybridizing to the nucleic acid sequence of a light-gated ion channel or connexin set forth above under stringent conditions, e.g., hybridization to filter-bound DNA in 0.5 M NaHPO4, 7% sodium dodecyl sulfate (SDS), 1 mM EDTA at 65° C., and washing in 0.1×SSC/0.1% SDS at 68° C. [29] and encodes a functionally equivalent gene product; or under less stringent conditions, such as moderately stringent conditions, e.g., washing in 0.2×SSC/0.1% SDS at 42° C., yet which still encodes a functionally equivalent light-gated ion channel or connexin protein.

In another aspect of the invention, variant light-gated ion channel or connexin polypeptides that differ in amino acid sequence by one or more substitutions, deletions, insertions, inversions, fusions, and truncations or a combination of any of these can be used in the methods of the present invention. Variant polypeptides can be fully functional or can lack function in one or more activities.

### Delivery of a Nucleic Acid to a Non-Excitable Cell

To create certain biological components of the present invention a nucleic acid encoding a light-gated ion channel is delivered to an implantable cell. In other embodiments, nucleic acids encoding two or more light-gated ion channels are delivered to the implantable cell. In certain embodiments a nucleic acid encoding a connexin is delivered to the implantable cell. In other embodiments, nucleic acids encoding two or more connexins are delivered to the implantable cell. In one embodiment, the nucleic acid is transfected using electroporation. Electroporation is a technique in which exposure of cells to a brief pulse of high voltage transiently opens pores in the cell membranes that allow macromolecules, such as DNA and proteins, to enter the cell.

Other methods of introducing genes into a cell include viral infection using, for example, adenovirus, adeno-associated virus (AAV), and lentivirus; liposome-mediated transfection (lipofection); transfection using a chemical transfection reagent; heat shock transfection; or microinjection. AAV, a small parvovirus associated with adenovirus, cannot replicate on its own and requires co-infection with adenovirus or herpesvirus in order to replicate. In the absence of helper virus, AAV enters a latent phase during which it stably integrates into the host cell genome. This latent phase makes AAV attractive for applications involving transfer of genes of up to about 4.4 kb, as the gene inserted into AAV can persist in the host cell genome for a long period [30]. Lentivirus, a member of the retroviral family, provides a potentially interesting alternative [31-32]. Unlike adenoviruses, electroporation and the use of lentiviral vectors allow persistent transgene expression without eliciting host immune responses.

### Delivery of Non-Excitable Cells to the Subject

The cell-based biological component of the present invention functions as a biological actuator converting the light from the optical stimulation unit to an electrical signal that can depolarize and/or repolarize cardiac tissue via gap junction connections with cardiomyocytes. Furthermore, for muscle cells (cardiac, skeletal or smooth muscle cells), the optical signal leads to electromechanical response, ending in mechanical contraction. Thus, the biological actuator converts the optical signal into mechanical contraction.

In one embodiment, the cell-based biological component is administered to one or more selected sites of the heart of a subject. Several methods to achieve focal delivery are feasible; for example, the use of catheters and needles, and/or growth on a matrix and a “glue.” Whatever approach is selected, the delivered cells should not disperse too far from the target site. Such dispersion could introduce unwanted electrical effects within the heart or in other organs. It is noteworthy that in a preliminary study involving injection of up to about 10⁶ hMSCs into the LV subepicardium of six adult dogs, nests of hMSCs were consistently found adjacent to the injection site but not at a distance [2].

In various embodiments, implantable cells are administered onto or into the heart by injection, catheterization, surgical insertion, or surgical attachment. The delivery site is determined at the time of administration, based on the subject's pathology, to give the optimal activation and hemodynamic response. Thus, the chosen site could be the sinoatrial (SA) node, Bachmann's bundle, the atrioventricular junctional region, His branch, left or right bundle branch, Purkinje fibers, right or left atrial muscle, or right or left ventricular muscle, the appropriate site being known to one of ordinary skill in the art.

In another embodiment, implantable cells are locally administered by injection or catheterization directly onto or into the heart. In further embodiments, the cell is systemically administered by injection or catheterization into a coronary blood vessel or a blood vessel proximate to the heart. In still further embodiments, the cell is injected onto or into an area of an atrium or ventricle of the heart. In other embodiments, the cell is injected onto or into the left atrium, a wall of a ventricle, a bundle branch of a ventricle, or the proximal LV conducting system of the heart.

The isoform or type of light-gated ion channel expressed in the non-excitable cells may also be changed depending on the delivery site. In addition, different levels of expression of the ion channel gene may be desirable in different delivery sites. Such different levels of expression may be obtained by using different promoters to drive expression at a desired level.

In certain embodiments, the biological component comprises between 5,000 to 1.5 million cells. In other embodiments the biological component comprises between about 700,000 to about 1.0 million cells. In one embodiment, the biological component comprises at least about 5,000 cells; at least about 10,000 cells; at least about 50,000 cells; at least about 100,000 cells; at least about 200,000 cells; at least about 500,000 cells; at least about 700,000 cells; or at least about 1 million cells.

In certain embodiments the number of cells administered to form the biological component is determined in terms of a ratio of donor cells delivered to cells in the target tissue (e.g., the ratio of non-excitable cells to cardiomyocytes). In one embodiment, the ratio is from about 1:10 to about 1:1000. In other embodiments the ratio of donor cells to target cells is from about 1:10 to about 1:20; from about 1:20 to about 1:30; from about 1:30 to about 1:40; from about 1:40 to about 1:50; from about 1:50 to about 1:60; from about 1:60 to about 1:70; from about 1:70 to about 1:80; from about 1:80 to about 1:90; from about 1:90 to about 1:100; from about 1:20 to about 1:100; from about 1:20 to about 1:500; from about 1:30 to about 1:70; from about 1:50 to about 1:100; from about 1:100 to about 1:200; from about 1:200 to about 1:300; from about 1:300 to about 1:400; from about 1:400 to about 1:500; and from about 1:500 to about 1:1000.

### Capable of Forming Gap Junctions

In one aspect, the invention provides a functional “tandem cell unit,” formed by a subject's target cell (e.g., a cardiomyocyte) and a non-excitable cell, acting as a donor for exogenous ion channels (e.g., channelrhodopsin2). For this unit to be functional (to fire an action potential upon light excitation), a proper gap junctional coupling is needed for closing the ion current loops.

Connexin 40 (Cx40), Connexin 43 (Cx43), and Connexin 45 (Cx45) are the major connexins expressed in the human heart. The gap junction channels formed by these connexins help maintain normal conduction velocity in the heart as the currents associated with action potential propagation move from myocyte to myocyte via gap junctions.

Thus, in one aspect the biological component of the optically-controlled biological device comprises a non-excitable cell expressing a light-gated ion channel protein and is capable of forming gap a junction channel with a cardiomyocyte when implanted in a subject. In one embodiment, the non-excitable cell endogenously expresses one or more connexin proteins. In one embodiment, the non-excitable cell has been engineered to express one or more connexins. In one embodiment, the non-excitable cell has been engineered to express one or more of Cx40, Cx43, and/or Cx45.

### Depolarizing/Repolarizing Signals

The light-gated ion channels when expressed in a cell affect the flow of ions across the cell membrane in response to light. The change in the ion flow corresponds to a change in the electrical properties of the cell such as the membrane potential. When the cell is coupled to an adjacent cell via a gap junction, the electrical properties of the adjacent cell are affected as well.

Cardiomyocytes have a negative membrane potential at rest. Stimulation above a threshold value induces the opening of voltage-gated ion channels allowing a flood of cations into the cell. The positively charged ions entering the cell cause the depolarization characteristic of an action potential. Following depolarization, a brief repolarization takes place with the efflux of potassium through fast acting potassium channels. Depolarization causes the opening of voltage-gated calcium channels and closing of the potassium channels and is followed by a titrated release of Ca²⁺ from t-tubules. This influx of calcium causes calcium-induced calcium release from the sarcoplasmic reticulum, and free Ca²⁺ causes muscle contraction. After a delay, slow acting potassium channels reopen and the resulting flow of K⁺ out of the cell causes repolarization to the resting state. Thus, repolarization is the return of the ions to their previous resting state, which corresponds with relaxation of the myocardial muscle.

In one embodiment, the cell expressing the light-gated ion channel (the donor cell) is stimulated by light causing the depolarization of one or more adjacent target cells (such as cardiomyocytes) via gap junctions connecting the donor cells to the target cells. In another embodiment, light is used to suppress depolarization in the target cells. In one embodiment, the cell expressing the light-gated ion channel (the donor cell) is stimulated by light causing the repolarization of one or more adjacent target cells (such as cardiomyocytes) via gap junctions connecting the donor cells to the target cells.

Manipulation of repolarization by light (when repolarization-related light-sesnitive ion channels are used, e.g. Halo, Arch, Mac) can alter the shape of the action potential (AP). In many pro-arrhythmic states, a disease-related AP prolongation takes place. Longer AP can be pro-arrhythmic by increasing the chances for early after-depolarization events (EAD)—abnormal secondary, undesired firings of APs. For example, class Ib and IV anti-arrhythmic drugs currently aim at shortening repolarization by manipulation of Na+ or Ca2+ ion channels. Optical suppression of such events (EADs) by light-forced or facilitated repolarization can be anti-arrhythmic without affecting contractile function (as in class IV Ca2+ channel blockers).

### Target Cell

One aspect of the invention features a donor cell engineered to express a light-gated ion channel protein, which forms a tandem cell unit with a target cell via gap junction channels between the donor and target cell. In one embodiment the target cell is a syncytial cell (i.e., a cell from a syncytial structure, such as the heart, bladder, liver, or gastrointestinal tract). In one embodiment the target cell is a cardiomyocyte. In other embodiments the target cell is a skeletal muscle cell or a smooth muscle cell.

### Optical Stimulation Unit

In one embodiment, the optical stimulation unit comprises a power supply, control circuitry, and one of more light sources. In further embodiments the optical stimulation unit also comprises one or more sensors.

The optical stimulating unit comprises one or more light sources. The light source can be any appropriate source of light capable of stimulating the light-gated ion channel expressed in the cells of the biological component. Such light sources include, but are not limited to, a laser, a laser diode, a light-emitting diode (LED), a organic light-emitting diode (OLED), an incandescent light source, an organic light source, or any other switchable light source with the ability to modulate the output at suitable rates. In one embodiment, the optical simulation unit comprises two or more light sources. In one embodiment, the optical stimulation unit comprises one or more optical fibers to deliver light to the biological component.

In one embodiment the optically-controlled biological device functions as a cardiac pacemaker. In one embodiment the optically-controlled biological device functions as a cardioverter/defibrillator. In one embodiment the optically-controlled biological device functions as both a cardiac pacemaker and a cardioverter/defibrillator.

In one embodiment the optical stimulating unit comprises one or more sensors that detect the status of one or more cardiac chambers.

The control circuitry (including programming) for cardiac pacing is known in the art and depends upon the type of cardiac pacing required. Pacemaker type is described by three (or four) letter code as described in Table 2.

TABLE 2
Pacemaker codes.

| Paced | Sensed | Response | Rate |
|---|---|---|---|
| A | A | T | R |
| V | V | I | |
| D | D | D | |

A: atrium;
V: ventricle;
D: dual;
T: triggered;
I: inhibited;
R: rate responsive.

The appropriate application to treat cardiac disease of the particular types of pacemakers is known to one of skill in the art.

The control circuitry (including programming) for ICDs is also known in the art. Such programming includes protocols for termination of ventricular tachycardia such as burst pacing (short bursts of paced beats delivered up to about 90% of the rate of the VT) and ramp pacing (short bursts of paced beats at a rate increasing up to about 90% of the rate of the VT).

The optical stimulation unit may use one or a combination of various methods known in the art to determine if an arrhythmia is normal or if it is ventricular tachycardia or ventricular fibrillation. For example, rate discrimination evaluates the rate of the ventricles and compares it to the rate in the atria. Rhythm discrimination determines the regularity of the ventricular tachycardia. Generally, ventricular tachycardia is regular. If the rhythm is irregular, it is usually due to conduction of an irregular rhythm that originates in the atria, such as atrial fibrillation. Morphology discrimination checks the morphology of each ventricular beat and compares it to a normally conducted ventricular impulse for the subject, which is often an average of multiple beats of the subject taken in the recent past.

In one embodiment the optically-controlled biological devices described herein are used in the treatment of a subject with a cardiac pacing disorder. Cardiac pacing disorders include, but are not limited to, cardiac arrhythmia, reentrant arrhythmia, bradycardia, tachycardia, sinus bradycardia, sinus tachycardia, ventricular tachycardia, supraventricular tachycardia, ventricular fibrillation, atrial flutter, atrial fibrillation, and pro-arrhythmic cardiac alternans.

In one embodiment the optically-controlled biological devices described herein functions as an optical-mechanical actuator modulating the function of target tissues including skeletal muscle and smooth muscle.

The use of cells expressing ChR2 as described herein demonstrates the feasibility of preparing light-activated biological pacemakers, but also demonstrates the formation of a tandem cell unit to influence the function of syncytial tissues. Thus, the device of the invention can also be used to deliver, for example, a signal to hyperpolarize smooth muscle (including vascular smooth muscle and bladder smooth muscle) inducing relaxation.

The term “subject” as used herein refers to any organism in need of treatment, or requiring preventative therapy, for cardiac disease with the methods and devices of the invention. In one embodiment the subject is a human.

It is to be understood and expected that variations in the principles of the invention disclosed herein may be made by one skilled in the art and it is intended that such modifications are to be included within the scope of the present invention.

Throughout this application, various publications are referenced. The disclosures of these publications in their entireties are hereby incorporated by reference into this application. The following examples further illustrate the invention, but should not be construed to limit the scope of the invention in any way.

### EXAMPLES

### Example 1

### Development and Characterization of a Cell Delivery System for Non-Viral Optogenetics

Plasmid Preparation:

The plasmid, pcDNA3.1/hChR2(H134R)-EYFP, was obtained from Addgene. It was expanded in bacteria (DH5a) on a LB+ampicillin agar plate overnight at 37° C. Selected colonies were further grown in LB-ampicillin medium with agitation, and the plasmid DNA was extracted into TE buffer using the Qiagen High Speed kit (Qiagen, Valencia, Calif.). Plasmid DNA (dsDNA) was quantified using the absorption ratio at 260 nm vs. 280 nm. After confirming the identity of the plasmid (by gel and spectrophotometry), it was stored at −20° C. in TE buffer at the obtained concentration (typically 2-4 μg/ml), and later diluted to 1 μg/ml for transfection.

Transfection:

HEK293 cells (ATCC, Manassas, Va.) were used as a model non-excitable cell and were transfected with the plasmid using Lipofectamine 2000 (Invitrogen) as directed: 4 μg of DNA and 10 μg of Lipofectamine in 250 μl medium for a 35 mm dish with cells. Gene expression was examined by EYFP signal the next day. 48 hours after transfection, cells were switched to selection medium, containing 500 μg/ml Geneticin (GIBCO Invitrogen). The selected cells with high fluorescence signal were maintained in Geneticin (500 μg/ml) containing culture medium at 37° C. in a humidified atmosphere incubator with 5% CO₂ and 95% air. Expanded HEK cell cultures showing near 100% expression were frozen at −80 C for later use. Immediately prior to use, the HEK-ChR2 cells were grown in DMEM (Dulbecco's Modified Eagle's Medium, GIBCO Invitrogen) supplemented with 10% FBS (fetal bovine serum, Sigma-Aldrich, St Louis, Mo.) and 1% penicillin-streptomycin (Sigma) at 37° C., 5% CO₂. Expression and functional properties were confirmed in passages 2 to 20 and used in co-culture experiments with cardiomyocytes.

Confirmation and Analysis of Light-Triggered ChR2-Current in the Cell Delivery System:

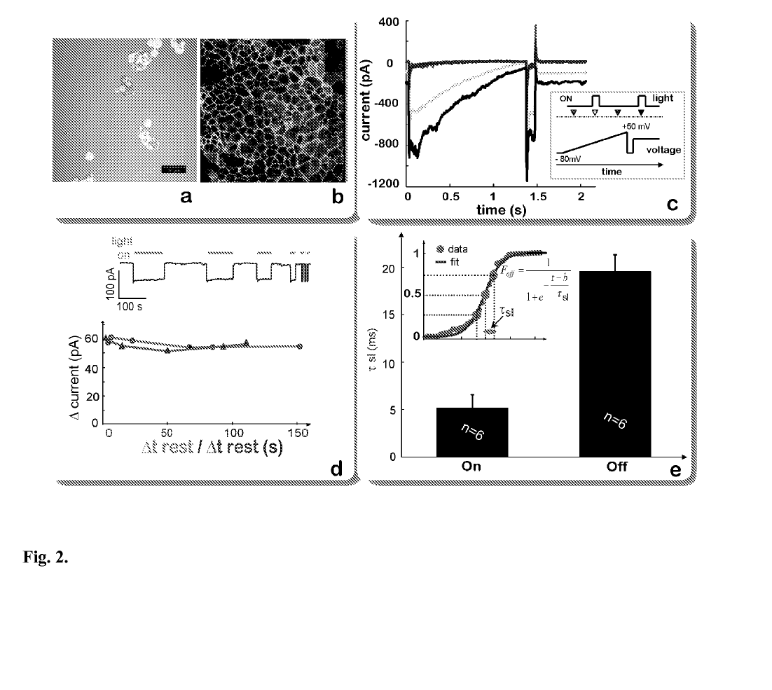

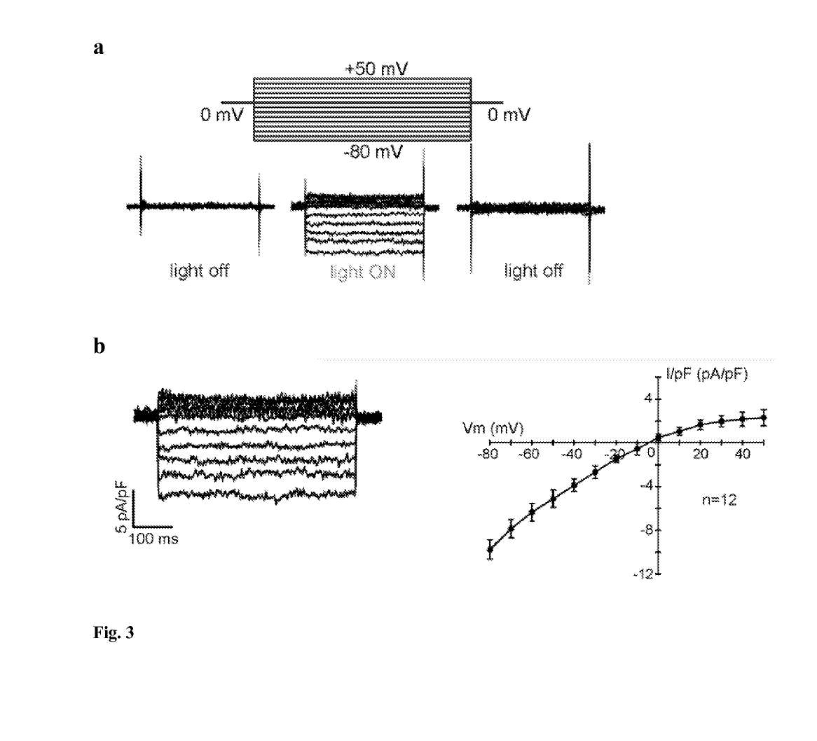

ChR2 cell membrane expression was confirmed in virtually 100% of the transfected HEK cells (FIG. 2b) using EYFP fluorescence as a marker. For functional measurements of the ChR2-current, the HEK-ChR2 cells were harvested by trypsinization, replated at low density on polylysine-coated coverslips and stored in DMEM medium at 37° in a humidified atmosphere incubator with 5% CO₂. The membrane current was recorded in single cells by whole-cell patch clamp with an Axopatch 1D amplifier (Axon instruments Inc, Foster City, Calif.). Borosilicate glass pipettes (World Precision Instruments Inc., Sarasota, Fla.) were pulled on a Flaming-Brown-type pipette puller (Sutter Instrument Co, Novato, Calif.) and heat-polished before use. Pipette resistances measured in Tyrode's solution were 3-4 MΩ when filled with pipette solution. The pipette solution contained (mmol/L) potassium aspartate 80, KCl 50, MgCl₂ 1, MgATP 3, EGTA 10 and HEPES 10 (pH 7.4 with KOH). The external solution contained (mmol/L) KCl 5.4, NaCl 140, MgCl₂1, CaCl₂ 1.8, HEPES 10 and Glucose 10 (pH 7.4 with NaOH). Membrane currents were recorded, digitized (DIGIDATA 1320A, Axon Instruments) and stored for offline analysis. There was a liquid junction potential of about 10 mV between the bath solutions and the electrode solution. The current was recorded as depolarizing 500 ms pulses from −80 mV to +50 mV with and without illumination (FIG. 3). The light-triggered ChR2 current was determined by subtracting the “off” light trace from the recorded response of light “on.” Illumination pulses were generated using the microscope-attached fluorescence light unit, filtered at 470 nm. The light-triggered inward ChR2-current was reproducible upon repeated on/off light pulses.

The kinetics of light-triggered ChR2-current was examined as the cells were clamped at −80 mV after obtaining whole-cell configuration, and light pulses of variable duration and spacing were applied sequentially. For the analysis of the current kinetics—activation and deactivation time-constants—nonlinear sigmoidal curve fit was applied to the rising and the falling portion upon light on/off pulse (FIG. 2d-e). The slope parameter (τₛₗ) was quantified.

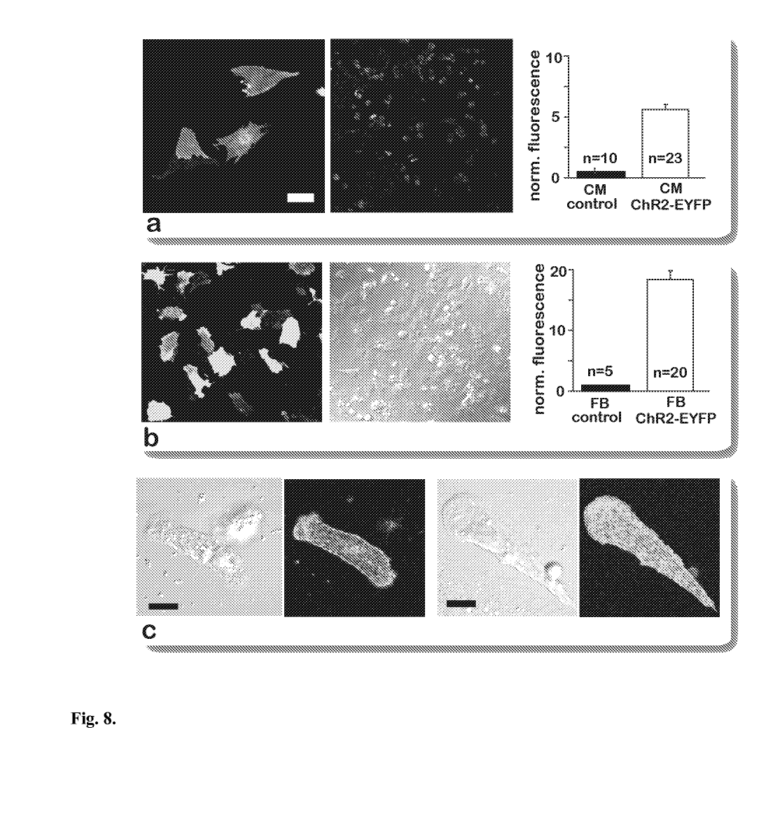

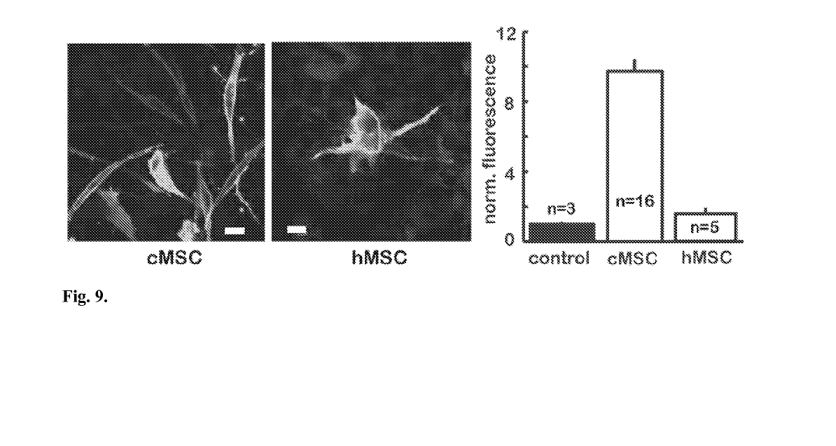

To validate the tandem cell unit (TCU) strategy for cardiac optogenetics, as a proof of principle, we developed a stable HEK cell line expressing a variant of ChR2. FIGS. 2 and 3 illustrate the properties of such donor cell line. Preserved expression and functionality were established for the HEK-ChR2 cell line after multiple freeze-thaw cycles and multiple passages (passages 2 to 30 were used for functional experiments) (FIG. 2b). Successful expression is possible in other non-excitable cell types, including mesenchymal stem cells that may yield more clinically relevant cell delivery systems (FIG. 8 and FIG. 9).

Confirmation of ChR2 functionality was done by whole-cell voltage clamp. Quantification of the steady-state light-sensitive ion current in single HEK-ChR2 cells (FIG. 3a-b) revealed that the channel is closed and non-contributing during dark periods regardless of transmembrane voltage, and has a mildly inwardly rectifying current-voltage (I−V) relationship when blue light is applied. Overall, significantly higher steady-state current densities were seen in our donor cells at all voltages in the I-V relationship, even at the low irradiance used here (0.24 mW/mm²), compared to previously reported light-induced ChR2 current in HEK cells [33], C. elegans muscle cells [34] or in cardiomyocytes [35]. For example, for comparable irradiance levels, at holding potential of −40 mV, about 20 times higher steady-state current densities were measured in our donor cells compared to ventricular myocytes from a transgenic mouse expressing ChR2 [35]. These data confirm high expression levels and/or functionality of ChR2 in the developed cell delivery system, important for optimizing light stimulation parameters.

Recent comprehensive characterization of ChR2 current kinetics indicates fast activation (<5 ms), deactivation (<10 ms) and inactivation (<50 ms) [33], thus making it suitable as excitatory (action potential—generating) current for cardiomyocytes during external optical pacing at relevant frequencies (5-12 Hz for rodents, 1-3 Hz for humans). Indeed, our kinetics characterization (FIG. 2d-e) estimates the activation and deactivation time constants for ChR2-mediated current in the ms range. Therefore suitable rates for cardiac pacing are attainable even without genetic modifications, as previously done for neural applications, where faster optogenetic tools in conjunction with much shorter action potentials allowed for pacing rates up to 200 Hz [36].

In contrast to the robust expression of ChR2 in HEK cells, much lower yield was seen when directly transfecting cardiomyocytes with ChR2 using nucleofector electroporation. This prevented direct synthesis of a large-scale ventricular syncytium from ChR2-expressing myocytes. Nevertheless, individual ChR2-expressing neonatal rat ventricular myocytes were excitable and contracting when optically stimulated and quiescent otherwise.

### Example 2

### Optically-Excitable Cardiac Syncytium: Primary Cardiomyocyte Cell Culture and Co-Culture with HEK-ChR2 cells

Primary Cardiomyocyte Cell Culture:

Neonatal Sprague-Dawley rats were sacrificed and cardiomyocytes were isolated by an approved Stony Brook University IACUC protocol as previously described [37]. Briefly, the ventricular portion of the hearts was excised and washed free of blood. The tissue was cut into small pieces and enzymatically digested with trypsin at 4° C. (1 mg/ml, USB, Cleveland, Ohio), then with collagenase at 37° C. (1 mg/ml, Worthington, Lakewood, N.J.) the next morning. Cardiac fibroblasts were removed by a two-stage pre-plating process. In some transfection experiments, these cardiac fibroblasts were used in conjunction with electroporation.

Co-Culture of Cardiomyocytes with HEK-ChR2 Cells:

Cardiomyocytes were plated onto fibronectin-coated glass coverslips at high density: 4×10⁵ cells/cm² for the control myocyte group and 3.5×10⁵ cells/cm² for the co-culture groups, mixed with approximately 7,700 or 3,500 HEK cells (for 45:1 and 100:1 initial plating ratios) onto glass bottom dishes in M199 medium (GIBCO Invitrogen) supplemented with 10% fetal bovine serum (GIBCO Invitrogen) for the first 2 days and then reduced to 2%. Cultures were maintained in an incubator at 37° C. with 5% CO₂ for 4 to 5 days before functional measurements.

Direct Expression of ChR2-EYFP in Neonatal Rat Cardiomyocytes and Cardiac Fibroblasts:

Freshly isolated neonatal rat cardiomyocytes were transfected by electroporation using a Nucleofector device (Amaxa Lonza, Gaithersburg, Md.) as follows: 4 μg of plasmid DNA was mixed with 100 μl of nucleofector solution for transfecting 4×10⁶ cells. Transfected cells were incubated in normal culture conditions. Similar conditions were used to transfect cardiac fibroblasts via electroporation. Expression of fluorescence was detected 24 to 48 hours after transfection using confocal fluorescence imaging.

Direct Expression of ChR2-EYFP in Mesenchymal Stem Cells:

Human mesenchymal stem cells (hMSC) were purchased from Clonetics/BioWhittaker, Walkersville, Md., USA, and cultured in mesenchymal stem cell growth medium—Poietics-MSCGM (BioWhittaker). Canine mesenchymal stem cells (cMSC) were isolated from the bone marrow of adult dogs and cultured in Poietics-MSCGM. Flow cytometry revealed 93.9% CD44+ and 6.1% cells were CD34+. Cells with spindle-like morphology were selected after flow cytometry characterization and replated for use. Transfected cells were incubated in normal culture conditions. Transfect via electroporation as described above. Expression of fluorescence was detected 24 to 48 hours after transfection using confocal fluorescence imaging.

Image Processing:

Background fluorescence was first subtracted for images of control (non-transfected) and transfected cells of the same type. Then the remaining integral fluorescence over identical areas was used to form a ratio (transfected/average control) in order to quantify and compare different cell types (FIG. 8 and FIG. 9).

Immunocytochemistry:

After 6 days of co-culture, samples were fixed for 10 min in freshly made 3.7% formaldehyde solution, followed by extensive washing in phosphate buffer solution (PBS). Cells were then permeabilized in a solution of PBS containing 0.2% Triton X-100 and 5% FBS for 5 min. The permeabilization solution was removed with 3×10 min washes in 1% FBS solution. Cells were then labeled simultaneously with mouse anti-connexin 43 (Invitrogen) for gap junctions and with rabbit anti-α-actinin (Sigma) primary antibodies diluted in 0.5% BSA solution, overnight at 4° C. The next day the cells were washed and then incubated in 0.5% BSA solution containing the appropriate secondary antibodies (Alexa Fluor 488 goat anti-rabbit IgG and Alexa 647 goat anti-mouse IgG) for 1 h, rinsed with 1% FBS solution, and then mounted in VectaShield (with DAPI) (Vector Laboratories) for imaging using the Olympus FluoView™ FV1000 imaging system with 60× oil lens (NA=1.42).

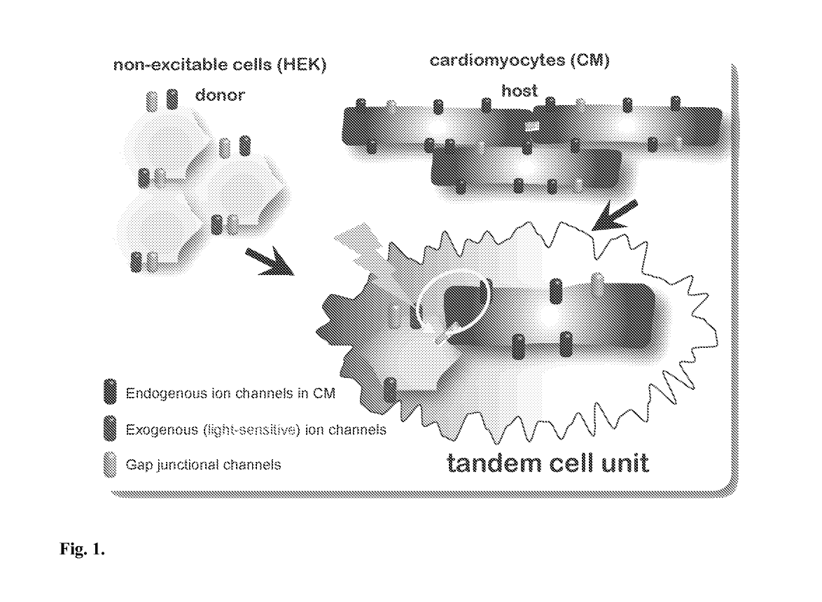

Here, we exploit this particular aspect (high coupling) characteristic for muscle tissues to develop a non-viral cell delivery system for expression of exogenous light-sensitive ion channels. FIG. 1 illustrates the concept of a functional “tandem cell unit” (TCU) formed by a host cardiomyocyte and a non-excitable cell, which is acting as a donor for exogenous ion channels, e.g. channelrhodopsin2. For this unit to be functional (to fire and action potential upon light excitation), a proper gap junctional coupling is needed for closing the ion current loops.

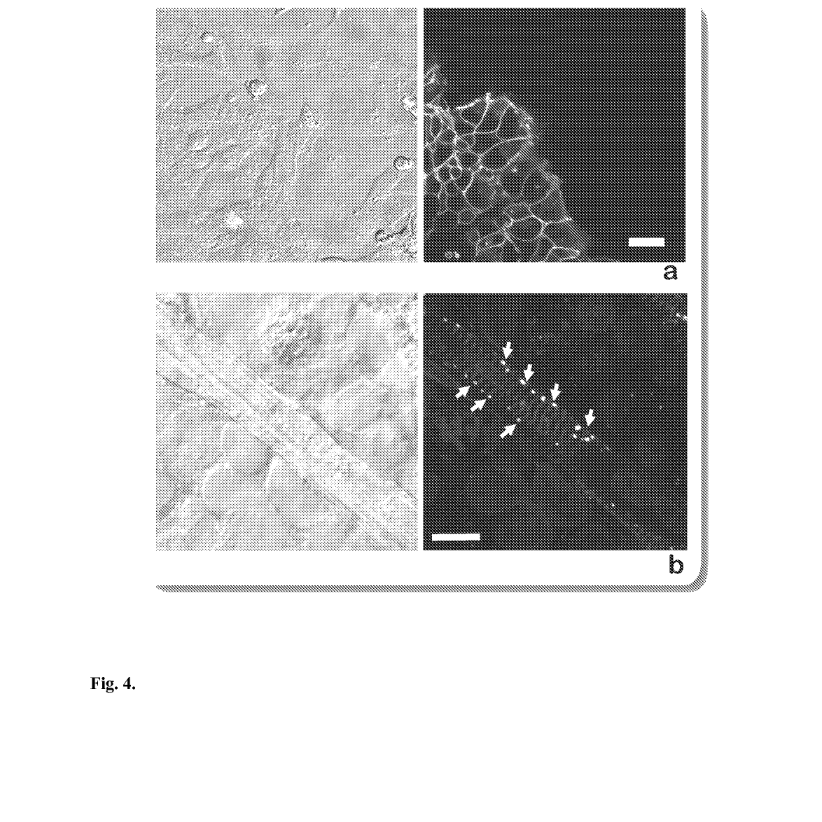

We used voltage clamp under blue light excitation (470/40 nm) to confirm functionality of ChR2—a large inward current was obtained. Co-cultures of neonatal rat cardiomyocytes and HEK cells (in ratio 50:1) were prepared to implement the “tandem cell unit” concept on a large scale (samples having >2 cm×2 cm area). Furthermore, immunocytochemistry confirmed the structural prerequisites for “tandem cell unit” formation, i.e., proximity and gap junctional coupling between the donor and host cells (FIG. 4).

### Example 3

### Demonstration of Tandem Cell Unit (TCU) Functionality in Cell Pairs of Adult Canine Ventricular Myocytes and HEK-ChR2

Adult mongrel dogs were euthanized as per IACUC protocol at Stony Brook University by intravenous injection of sodium pentobarbitone (80 mg/kg body weight) and the heart was removed. Canine ventricular cells were isolated using a modified Langendorff procedure [38] perfusing a wedge of the left ventricle through a coronary artery with 0.5 mg ml-1 collagenase (Worthington) and 0.08 mg ml⁻¹ protease (Sigma) for 10 min before tissue digestion. Prior to plating, isolated cardiomyocytes were stored in Kraft-Brühe (KB) solution (in mM: KCl, 83; K₂HPO₄, 30; MgSO₄, 5; Na-Pyruvic Acid, 5; β-OH-Butyric Acid, 5; Creatine, 5; Taurine, 20; Glucose, 10; EGTA, 0.5; KOH, 2; and Naₑ-ATP, 5; pH was adjusted to 7.2 with KOH) at room temperature. The canine ventricular myocytes were plated onto laminin-coated glass coverslips (10 μg/ml, Invitrogen) and incubated at 37° C. to ensure attachment. HEK-ChR2 cells were added within 24 h at low density to stimulate formation of individual cell pairs and the co-culture was maintained in Medium 199 (Gibco) supplemented with 15% FBS, 2 mM 1-glutamine, 100 U ml⁻¹ penicillin, 100 μg ml⁻¹ streptomycin and 50 μg ml⁻¹ gentamicin.

Dual patch clamp experiments were performed within 48 hours after plating. Briefly, experiments were carried out on heterologous (HEK-ChR2— canine myocyte) cell pairs within 48 hours after plating, as described previously [39]. A dual whole-cell voltage-clamp method was used to control and record the membrane potential of both cells and to measure associated membrane and junctional currents [39-40]. Each cell of a pair was voltage clamped at the same potential by two separate patch clamp amplifiers (Axopatch 200, Axon Instruments). To record junctional conductance, brief voltage steps (±10 mV, 400 ms) were applied to one cell of a pair, whereas the other cell was held at constant voltage and the junctional currents were recorded from the unstepped cell. Membrane and action potentials were recorded in current-clamp mode.

For electrical recordings, glass coverslips with adherent cells were transferred to an experimental chamber mounted on the stage of an inverted microscope (Olympus-IX71) equipped with a fluorescence imaging system. The chamber was perfused at room temperature (˜22° C.) with bath solution containing (in mM): NaCl, 140; MgCl₂, 1; KCl, 5; CaCl₂, 2; HEPES, 5 (pH 7.4); glucose, 10. Perfusion with 200 μM of carbenoxolone (Sigma) was used to block cell-cell communication. The patch pipettes were filled with solution containing (in mM): K⁺ aspartate⁻, 120; NaCl, 10; MgATP, 3; HEPES, 5 (pH 7.2); EGTA, 10 (pCa ˜8). Patch pipettes were pulled from glass capillaries (code GC150F10; Harvard Apparatus) with a horizontal puller (DMZ-Universal, Zeitz-Instrumente). When filled, the resistance of the pipettes measured 1-4MΩ.

Immunostaining of Co-Culture:

For immunocytochemistry, the co-cultures were fixed and permeabilized with 3.7% formaldehyde and 0.02% Triton-X 100 before being stained with a monoclonal mouse antibody against sarcomeric α-actinin (Sigma). Samples were visualized using goat anti-mouse antibody conjugated with fluorophore Alexa 546 (Invitrogen) and imaged on the Olympus FluoView confocal system.

Western Blots of Cx43 and α-Tubulin:

Cells from three groups (HEK293, HEK+ChR2 and stably transfected HeLa+Cx43 cells) were collected, lysed and centrifuged to obtain a pellet. The pellets were re-suspended in cold RIPA buffer (R0278, Sigma), protease Inhibitor cocktail (P2714, Sigma), sodium orthovanadate (S-6508, Sigma) and PMSF (P-7626, Sigma); after centrifugation, the supernatants were transferred to pre chilled microtubes. Protein concentration for each sample was determined by the Bradford assay. Total protein of 30 micrograms from each lysate was mixed with equal volume of laemmli sample buffer (161-0737, Bio-Rad, Hercules, Calif.) containing β-mercaptoethanol and boiled for 5 minutes at 95° C. After centrifugation, samples were loaded on a SDS-polacrylamide gel. MagicMark XP Protein Standard (LC5602, Invitrogen) was loaded along with the samples. After separation by electrophoresis at 115 V for 90 minutes in tris-glycine/SDS buffer, proteins were transferred to immobilon-P membrane (Millipore, Billerica, Mass.) by electrophoresis at 100 V for 60 minutes in tris-glycine/methanol buffer. Nonspecific antibody binding was blocked for 1 hour by 5% blotting grade blocker non-fat dry milk (Bio-Rad) dissolved in 1×TBST. The following antibodies were used: primary anti-Cx43 antibody raised in rabbit (C 6219, Sigma), secondary goat-anti-rabbit antibody (sc-2004, Santa Cruz); a primary antibody for α-tubulin at 55 kD (sc-8035, Santa Cruz), and a secondary goat-anti-mouse antibody for tubulin from Pierce, Rockford, Ill. The secondary antibodies were detected using SuperSignal West Femto Maximum Sensitivity Substrate (34095, Pierce) and images obtained by exposing the membrane to HyBlot CL autoradiography film. Quantification of the Cx43 bands relative to the α-tubulin bands was done using a buil-in routine in ImageG.

Carbenoxolone Treatment to Test Effects of Cell Coupling on Tandem Cell Units:

Carbenoxolone, CBX, (Sigma), a gap junctional uncoupler [39], was used at a concentration 200 μM in the dual-patch experiments with canine cardiomyocytes and HEK-ChR2 or in the cardiac syncytium of neonatal rat cardiomyocytes and HEK-ChR2. In the latter case, CBX was applied for 20 min (without perfusion) in the co-cultures of HEK-ChR2 cells and cardiomyocytes. Contractility movies were recorded in response to optical pacing before and during administration of carbenoxolone, and upon washout to assess the role of gap junctional coupling in the functionality of the tandem cell unit.

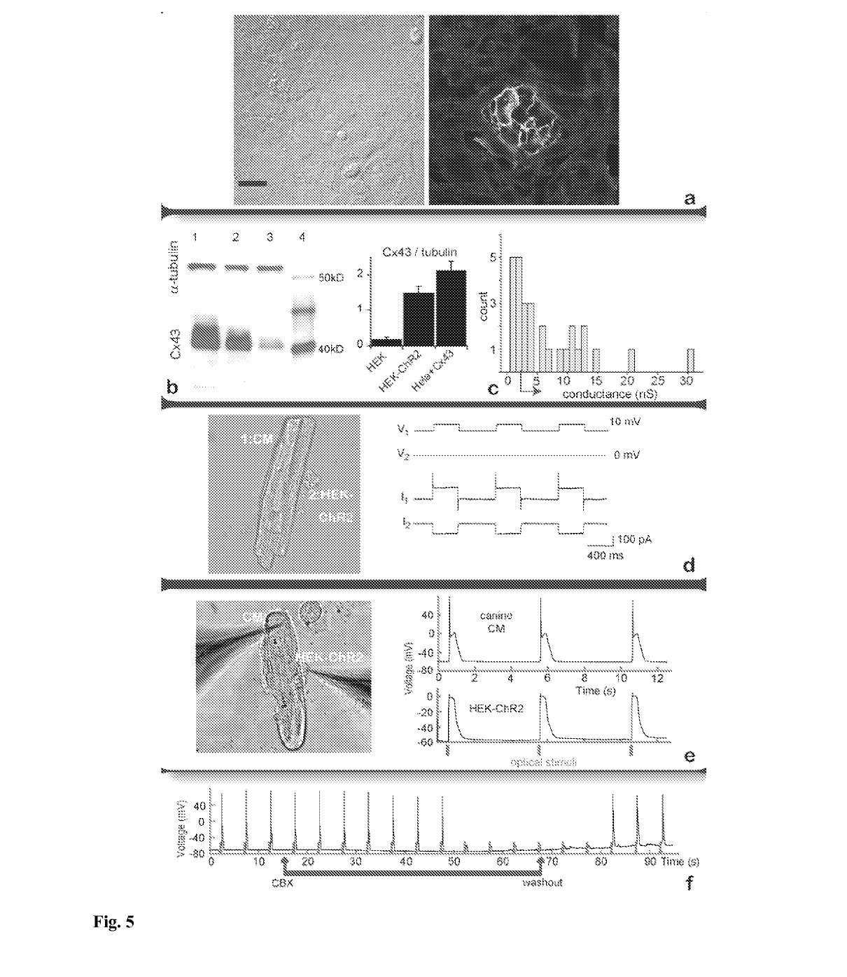

The TCU approach for cardiac optogenetics, i.e., inscribing light-sensitivity into cardiomyocytes and cardiac tissue without their direct genetic modifications, was validated in cell pairs of CM and HEK-ChR2 cells, as well as in a synthesized large-scale cardiac syncytium. ChR2-expressing donor cells were most often found to aggregate in small clusters among the neonatal CMs rather than disperse as single cells (FIG. 5a). The donor cells expressed a significant amount of Cx43, as confirmed by a Western blot (FIG. 5b). Substantially more Cx43 protein was seen in the HEK-ChR2 cells compared to the parental cell line without ChR2.

Functional response of TCUs to optical stimulation was confirmed in cell pairs of adult canine CM and HEK-ChR2 cells using dual-clamp to estimate coupling (FIG. 5c-d) and to record light-triggered action potentials in the cardiomyocytes (FIG. 5e-f). A histogram of measured coupling in spontaneously formed cell pairs (n=31) of canine CM and HEK-ChR2 over 48 hour period is shown (FIG. 5c). A robust response was seen in a wide range of coupling values spanning an order of magnitude, starting as low as 1.5 nS. Interestingly, a similar low critical coupling value (1.5-2 nS), below which TCU functionality failed, was found previously in the generation of a two-cell pacemaking unit by a donor cell carrying HCN2 (a gene encoding for the pacemaking current I_{f}) and a cardiomyocyte [39]. Extreme uncoupling abolished the light-sensitivity of the myocytes in the TCUs—values below 1.5 nS and pharmacological uncoupling with cabenoxolone provided further proof for gap junctions' role in the TCU functionality for neonatal rat and for adult canine myocytes (FIG. 5f).

In a functional TCU pair, the cardiomyocytes generated normal action potentials upon stimulation by blue light (470 nm, 0.13 mW/mm², 10 ms pulses) indistinguishable from electrically-triggered ones (FIG. 5e). The donor cell's membrane potential followed passively by a low-pass filtered version of an action potential (FIG. 5e). In a spatially-extended (several centimetres) two-dimensional cardiac syncytium of randomly mixed neonatal rat CMs and HEK-ChR2 (45:1 initial plating ratio), robust synchronous contractions were registered upon stimulation by blue light 2-3 days after plating.

### Example 4

### Ultra-High Resolution Optical Mapping of Cardiac Excitation Waves Triggered by Light in Co-Cultures

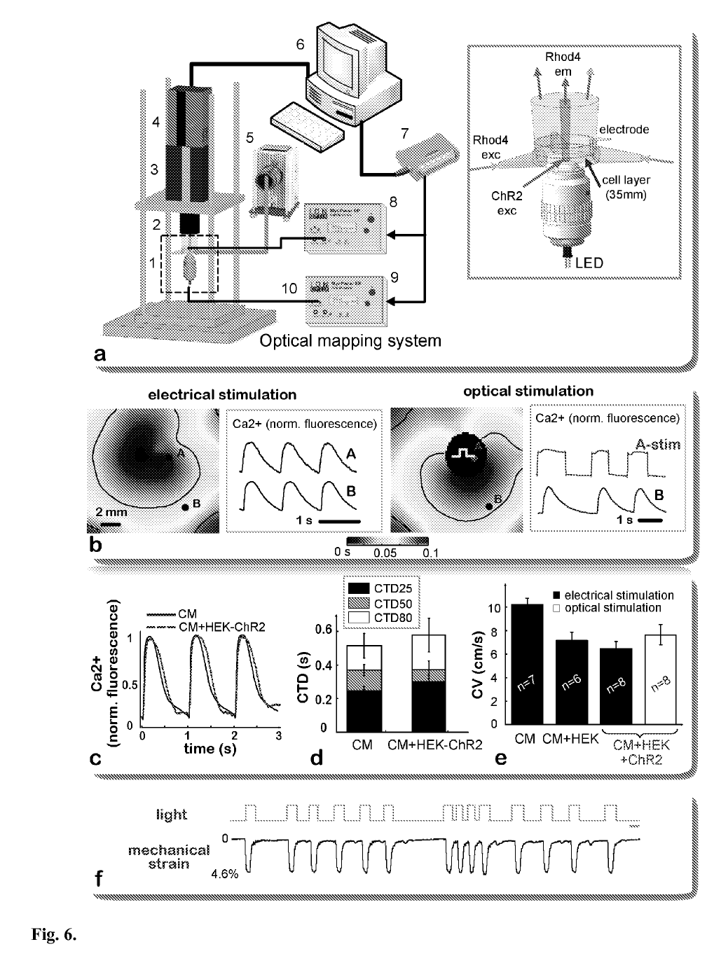

Two-dimensional optical mapping over a large field of view (about 2.2 cm) was done with a custom-developed macroscopic system [37, 38] allowing for ultra-high spatio-temporal resolution. The system (FIG. 6a) includes a CMOS camera (pco, Germany) recording images at 200 frames per second (fps) over 1,280×1,024 pixels), an Gen III fast-response intensifier (Video Scope International, Dulles, Va.), collecting optics (Navitar Platinum lens, 50 mm, f/1.0) and filters, excitation light source (Oriel with fiber optics lights guides) and an adjustable imaging stage. Subcellular spatial resolution was achieved—about 22 μm per pixel. All measurements were done in normal Tyrode's solution at room temperature. Quest Rhod-4 (AAT Bioquest, Sunnyvale, Calif.) was used to label the cells for tracking Ca²⁺ waves. This optical dye was chosen for wavelength compatibility with ChR2 and EYFP excitations/emissions. Excitation light (525 nm) for Ca²⁺ recording was delivered through non-conventional distributed tangential illumination (90° angle with respect to the optical axis) to accommodate optical stimulation but also to achieve superior contrast by complete uncoupling of the Rhod-4 excitation from the light-gathering optics (FIG. 6a). Excitation light for Rhod-4 was provided by a QTH lamp with a branching liquid light guide, attached to a custom designed experimental chamber with reflective inner walls and open bottom surface, accommodating a 35 mm dish with the sample. Emitted Rhod-4 fluorescence was collected at 585 nm through an emission filter in front of the intensified camera on top of the sample.

Movies of propagation were acquired using Cam Ware (pco, Germany) data acquisition software. Raw data were binned (2×2) and analyzed in custom-developed Matlab software to extract quantitative information about calcium transient morphology, conduction velocity etc. Activation maps (based on time of maximum rise in Ca²⁺) and phase movies (using the Hilbert transform) were generated after filtering spatially (Bartlett filter, 5-pixel kernel) and temporally (Savitsky-Golay, order 2, width 7) [37, 41].

Electrical and Optical Pacing:

For records of electrically-triggered activity, cells were paced by Pt electrodes, connected to a computer-driven Myopacer stimulator (IonOptix, Milton, Mass.). Excitation pulses for light-triggered activity were delivered through the bottom of the dish from an optically focused light from an LED (470 nm, 1.35 cd, 20 mA, 5 mm, 30 deg angle) from Optek Technology (Carrollton, Tex.) or from a fiber-optics coupled high-power blue LED (470 nm, 1.6 A), Thor Labs (Newton, N.J.), connected to the TTL output of a second computer-driven Myopacer stimulator. Irradiance (in mW/mm²) was measured at the cell monolayer site using a Newport digital optical power meter Model 815 (Newport, Irvine, Calif.), with a sensor area of 0.4 cm×0.4 cm, with wavelength set at 470 nm.

Recording Light-Triggered Contractions:

Microscopic imaging for confirmation of gene expression or for documenting contractions by optical excitation was done with the Olympus FluoView™ FV1000 confocal system at room temperature. An EMCCD camera (ImagEM EMCCD camera from Hamamatsu, Bridgewater, N.J.) attached to the Olympus FluoView™ FV1000 microscope was used to record contractility movies at 20 fps with 60× oil lens (NA=1.42) using SlideBook 5 software (Intelligent Imaging Innovations, Denver, Colo.). In addition, contractility response was also documented by automatic optical tracking of cell length [42] at 250 Hz using an IonOptix videosystem (IonOptix) attached to a Nikon TE2000 inverted microscope.

Synchronized wave propagation is essential for normal functionality of the heart and efficient mechanical contraction. Lethal arrhythmias occur when the generation or propagation of these excitation waves is adversely altered (failure to initiate, abnormal propagation velocity and/or path). Hence, arrhythmias are complex spatio-temporal phenomena and require matching imaging tools to dissect. We have developed ultrahigh resolution optical mapping system, allowing for measurements of propagating voltage and calcium waves using fast fluorescence indicator dyes over centimeter-scale with subcellular resolution [41]. Excitation waves were triggered by a computer-controlled light source (Xenon lamp with a liquid light guide, filtered at 470/40 nm and focused under the sample at a circular spot with diameter of 4 mm). Interestingly, irradiance (0.9 mW/cm²) for supra-threshold stimulation was 2-3 orders of magnitude lower than previously reported for use in neurons with ChR2. This very low intensity of light needed, is an important factor in confirming a lack of heat-related effects, no phototoxicity, and in considering future implantable devices. The discrepancy with the reported light levels for neurons and cardiomyocyte monolayers here, may stem from different cellular properties, single vs. multicellular preparations and different focusing of the light. Optical mapping of calcium waves triggered by localized electrical and optical stimulation in the same sample, revealed similar conduction velocities and calcium transient morphologies (away from the stimulus site) for both types of stimulation, thus confirming equivalent triggering abilities for both (FIG. 6). As controls, we imaged pure cardiomyocyte cultures and co-cultures of cardiomyocytes and HEK cells not expressing the ChR2. While electrical wave propagation was similar among the three imaged groups, the controls never produced excitation in response to the light triggers. Considering the electromechanical nature of cardiomyocytes, this demonstrates a direct light-triggered muscle contraction, illustrating a proper excitation-contraction coupling in this hybrid cardiac tissue (FIG. 6). This demonstration of mechanical cellular action triggered by light-sensitive ion channels opens the door to further development of light-driven biological actuators with highly efficient energy transfer—a new class of biological micro-opto-electro-mechanical systems (BioMOEMS). It also confirms feasibility for light control in other muscle cells and tissue, including applications to areas of prosthetics, respiratory control bowel movement, incontinence, etc.

Synthesized optically-excitable cardiac tissue was subjected to functional testing. Synchronized wave propagation is essential for the heart's normal functionality and efficient mechanical contraction; lethal arrhythmias occur when the generation or propagation of these excitation waves is altered (failure to initiate, abnormal propagation velocity and/or path). Accordingly, we have developed an ultra-high resolution optical mapping system [41, 37] to dissect cardiac wave propagation during external pacing or arrhythmic activity over a centimeter-scale (>2 cm) with subcellular resolution (22 μm/pix) at 200 fps using fast voltage and calcium-sensitive dyes [41]. This optical mapping system was made compatible with simultaneous optical excitation (FIG. 6a) so that excitation light for the fluorescence measurements did not induce ChR2 excitation and ChR2 excitation did not interfere with the measurements. While mapping was done here with Rhod-4, a calcium-sensitive fluorescent dye, suitable voltage-sensitive probes with similar spectral properties can also be used, e.g. di-4 or di-8-ANEPPS [41]. In normal pacing conditions, cardiac calcium transients are an excellent surrogate for action potentials, and calcium dyes outperform voltage-sensitive dyes in signal-to-noise ratio.

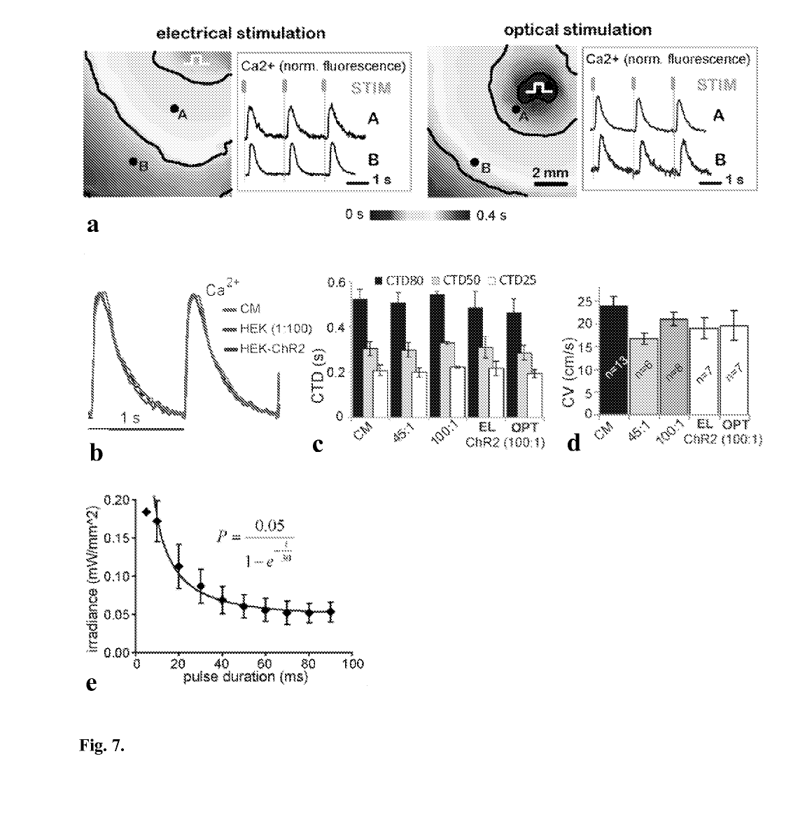

Optical mapping of propagating waves triggered by localized electrical and optical stimulation in the same sample, revealed similar conduction velocities and calcium transient morphologies, thus confirming equivalent triggering abilities for both modes of stimulation (FIG. 7a-b). Pure cardiomyocyte cultures and co-cultures of cardiomyocytes and HEK cells without ChR2 served as controls. At the mixing ratios used here, the presence of HEK cells, with or without ChR2, did not alter the recorded calcium transients (FIG. 7b-c, see also FIG. 6c-d), p=0.36 with ChR2, p=0.44 without ChR2. However, the mixing ratio was a significant factor (p<0.0001) in modulating CV, as revealed by a two-way ANOVA, i.e about 30% drop in CV was seen at initial plating ratios of 45:1 (CM:HEK) while a ratio of 100:1 lead to a smaller (non-significant, p=0.06) reduction (FIG. 7d). The presence of ChR2 did not contribute as a significant factor in CV modulation (p=0.16), even though a slight trend to a decrease was seen. Further titration (higher mixing ratios) and/or localized spatial distribution are likely to minimize these effects. Electrical and optical pacing in light-sensitive samples (CM:HEK+ChR2) resulted in identical wave propagation properties. The controls (CM only and CM+HEK without ChR2) were quiescent and never produced excitation in response to light triggers.

The response of the syncytium to optical excitation was captured by constructing a strength-duration curve, describing minimum irradiance over a range of pulse duration values for a point excitation of the 2D syncytium (2 mm fiber-optic coupled controllable LED) (FIG. 7e). The fitted curve revealed a particularly low minimal irradiance levels (average rheobase [43] for excitation of about 0.05 mW/mm²)—at least an order of magnitude lower than previously shown values for optical stimulation of ventricular or atrial tissue [35]. Within the tested diameter for light delivery, macroscopic excitability remained uniform across the tissue.

Considering the electromechanical nature of cardiomyocytes, we also show direct light-triggered muscle contraction, confirming intact excitation-contraction coupling in single myocytes or hybrid cardiac tissue (FIG. 6f). This demonstration of mechanical response triggered by light-sensitive ion channels suggests possible development of light-driven actuators with efficient energy transfer and illustrates the feasibility for direct optogenetic control in other muscles.

Energy Needs in Cardiac Optogenetics:

Previous studies in neuroscience have reported optical energies used to excite single neurons or brain tissue [8, 44, 45] in a wide range of high values (approximately 8 to 75 mW/mm²). The well-coupled spatially-extended cardiac tissue was expected to present higher load for optical stimulation, thus possibly requiring even higher irradiance values. Yet, surprisingly, in Bruegmann et al.'s study [35], significantly lower light levels (0.5 to 7 mW/mm²) were sufficient to optically stimulate cardiac tissue in vitro or in vivo for a wide range of pulse durations.

In our TCU approach, during optical pacing in the cell pairs or in the two-dimensional cardiac syncytium, we measured irradiance at 470 nm as low as 0.006 mW/mm². The strength-duration curve (FIG. 7e) further corroborated low irradiance needed across pulse durations. Interestingly, this is much lower than reported for neuroscience applications and about 1-2 orders of magnitude lower than previously reported values for cardiac excitation in cells and tissue [35] for comparable pulse durations. The significantly lower optical energy needed in our study can be explained, at least partially, by the superior light sensitivity of the donor cells as seen in the higher ChR2 current densities (FIG. 3b).

### Example 5

### Equivalent Circuit Analysis of TCU

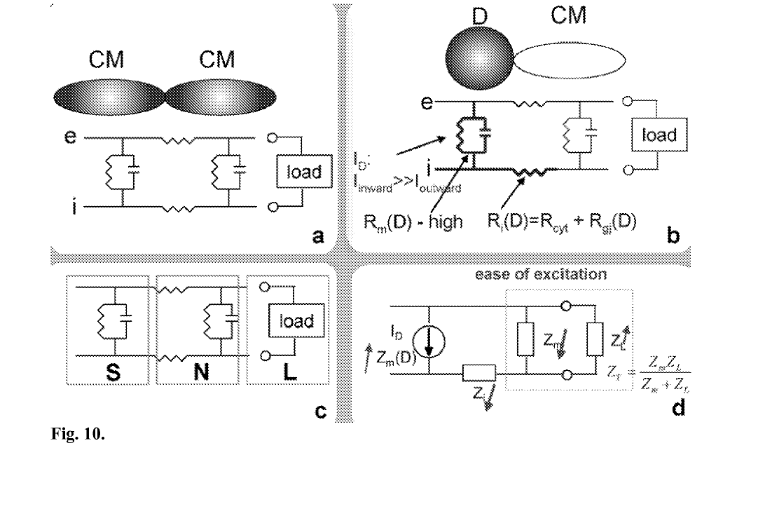

When comparing the TCU strategy to direct expression of ChR2 in the native myocytes (mostly by viral methods) there are at least several important differences (FIG. 10): (1) the donor (D) cells are non-excitable and typically do not have major repolarizing currents, i.e., the ChR2 inward current is their main current, unlike native CMs; (2) the D-cells have higher membrane impedance at rest due to smaller/negligible inward rectifier, I_{K1}, and typically have a more depolarized resting potential; (3) compared to CM-CM coupling, the D-CM coupling is typically somewhat reduced.

For a spatially-extended tissue, when the cell pair (TCU) is connected to some “load” of excitable cells (CMs), the system can be abstracted to a Source-Neighbor-Load (S-N-L) triad for easier analysis. A simplified equivalent circuit of such a triad is presented in FIG. 10d, derived under the following assumptions: use of equivalent impedances (Z); assuming negligibly small extracellular impedance Zₑ; for the immediate neighbor cells to be charged (N), we consider the membrane Zₘ and intracellular (coupling) impedance Zᵢ, but for the rest of the tissue—consider a lumped/load impedance, Z_{L}. Arrows indicate the direction of contribution of the different circuit elements towards “ease of excitation,” as analyzed below.

For the simplified circuit in FIG. 10d, we can derive the following expressions, representing the underlying current-divider circuits and the total current drawn by the circuit, I_{T}, as well as the current to excite the immediate neighbors, i.e., the current of interest, Lₘ:

$$\begin{matrix}{I}_{T} = \frac{{Z}_{m} ( D )}{{Z}_{i} + {Z}_{T}} {I}_{D} & ( 1 ) \\ {I}_{m} = \frac{{Z}_{L}}{{Z}_{m} + {Z}_{L}} {I}_{T} & ( 2 )\end{matrix}$$

In determining how the elements of the circuit determine the “ease of excitation” of the system, i.e what fraction of I_{D} does Iₘ constitute, our simplified analysis only considers steady-state passive response and ignores the kinetics of the ion channels and/or transient processes, which are admittedly very important.

The following observations can be made:

1) Higher Zₘ for the source, i.e., Zₘ(D)>Zₘ(CM), makes the source closer to an “ideal current source” (with infinite internal resistance/impedance) and therefore increases the “ease of excitation”, i.e., for a given light-induced I_{D}, more current Iₘ will be available for excitation of the neighbors in the TCU case, which follows directly from Eqn (1).

2) Higher load, i.e., low Z_{L}, such that Zₘ>>Z_{L}, and Z_{T}≈Z_{L}→0, that may occur in very well-coupled, spatially-extended tissue (higher dimensionality) will indeed make it harder to excite by making Im a smaller fraction of ID—see simplified combination of Eqns (1) and (2) for this case:

$${I}_{m} = \frac{{Z}_{m} ( D )}{{Z}_{i}} \frac{{Z}_{L}}{{Z}_{m}} {I}_{D} \qquad ( 3 )$$

3) Very low load, i.e., high Z_{L}, such that Z_{L}>Zₘ, and Z_{T}≈Zₘ) applies for small cell clusters and/or highly uncoupled tissue, and highlights the importance of the coupling to the immediate neighbors, Zᵢ and the membrane impedance of the immediate neighbors. The ease of excitation will improve by higher coupling to the neighbors for CM-CM (low Zᵢ) in this case compared to D-CM and lower Zₘ of the neighbors (possibly determined by the equivalent K⁺ conductance in the neighbor CMs), as seen from the simplified Eqn. (4).

$${I}_{m} = \frac{{Z}_{m} ( D )}{{Z}_{i} + {Z}_{m}} {I}_{D} \qquad ( 4 )$$

4) Overall, better coupling for S-N, but reduced coupling (and equivalent conductivity) for N-L works towards ease of excitation. It is important to note, however, that for spatially distributed D-cells, the S-N coupling in the S-N-L triad plays a dual role, i.e., it affects both the S-N charge transfer and the equivalent “load” presented by the tissue. All parameters indicated in d act in parallel and actual simulations, quantification is needed to fully uncover the extent of their individual contribution.

### REFERENCES

1. Liechty K. W., et al. Human mesenchymal stem cells engraft and demonstrate site-specific differentiation after in utero transplantation in sheep. Nat Med. 2000. 6: 1282-1286.
2. Plotnikov A. N., et al. Adult human mesenchymal stem cells carrying HCN2 gene perform biological pacemaker function with no overt rejection for 6 weeks in canine heart. Circulation. 2005. 112: II-221 (Abstract).
3. Berthold, P., et al., Channelrhodopsin-1 Initiates Phototaxis and Photophobic Responses in Chlamydomonas by Immediate Light-Induced Depolarization. Plant Cell. 2008. 20(6):1665-1677.
4. Nagel, G., et al., Channelrhodopsin-1: A Light-Gated Proton Channel in Green Algae. Science. 2002. 296(5577):2395-2398.
5. Nagel G Fau-Szellas, T., et al., Channelrhodopsins: directly light-gated cation channels. Biochem Soc Trans. 2005. 33(Pt 4):863-6.
6. Nagel G Fau-Szellas, T., et al., Channelrhodopsin-2, a directly light-gated cation-selective membrane channel. Proc Natl Acad Sci USA. 2003. 100(24):13940-5.
7. Ritter, E., et al., Monitoring Light-induced Structural Changes of Channelrhodopsin-2 by UV-visible and Fourier Transform Infrared Spectroscopy. Journal of Biological Chemistry. 2008. 283(50):35033-35041.
8. Cardin, J. A., et al., Targeted optogenetic stimulation and recording of neurons in vivo using cell-type-specific expression of Channelrhodopsin-2. Nat. Protocols. 2010. 5(2):247-254.
9. Douglass, A. D., et al., Escape Behavior Elicited by Single, Channelrhodopsin-2-Evoked Spikes in Zebrafish Somatosensory Neurons. Current Biology. 2008. 18(15):1133-1137.
10. Zhang, F., et al., Channelrhodopsin-2 and optical control of excitable cells. Nat Meth. 2006. 3(10):785-792.
11. Zhang, F., et al., Red-shifted optogenetic excitation: a tool for fast neural control derived from Volvox carteri. Nat Neurosci, 2008. 11(6):631-633.
12. Kianianmomeni, A., et al., Channelrhodopsins of Volvox carteri Are Photochromic Proteins That Are Specifically Expressed in Somatic Cells under Control of Light, Temperature, and the Sex Inducer. Plant Physiol. 2009. 151(1):347-366.
13. Zhang, F., et al., Optogenetic interrogation of neural circuits: technology for probing mammalian brain structures. Nat. Protocols. 2010. 5(3):439-456.
14. Zhao, S., et al., Improved expression of halorhodopsin for light-induced silencing of neuronal activity. Brain Cell Biology. 2008. 36(1):141-154.
15. Gradinaru, V., K. Thompson, and K. Deisseroth, eNpHR: a Natronomonas halorhodopsin enhanced for optogenetic applications. Brain Cell Biology. 2008. 36(1):129-139.
16. Chow, B. Y., et al., High-performance genetically targetable optical neural silencing by light-driven proton pumps. Nature. 2010. 463(7277):98-102.
17. Han, X. and E. S. Boyden, Multiple-Color Optical Activation, Silencing, and Desynchronization of Neural Activity, with Single-Spike Temporal Resolution. PLoS ONE. 2007. 2(3):e299.
18. Zhang, F., et al., Multimodal fast optical interrogation of neural circuitry. Nature. 2007. 446(7136): p. 633-639.
19. Ihara, K., et al., Evolution of the archaeal rhodopsins: evolution rate changes by gene duplication and functional differentiation. Journal of Molecular Biology. 1999. 285(1):163-174.
20. Waschuk, S. A., et al., Leptosphaeria rhodopsin: Bacteriorhodopsin-like proton pump from a eukaryote. Proc. Natl. Acad. Sci. USA. 2005. 102(19):6879-6883.
21. Bowie et al., Science. 1990 247:1306-1310.
22. Computational Molecular Biology, Lesk, A. M., ed., Oxford University Press, New York, 1988.
23. Biocomputing: Informatics and Genome Projects, Smith, D. W., ed., Academic Press, New York, 1993.
24. Computer Analysis of Sequence Data, Part 1, Griffin, A. M., and Griffin, H G., eds., Humana Press, New Jersey, 1994.
25. Sequence Analysis in Molecular Biology, van Heinje, G., Academic Press, 1987.
26. Sequence Analysis Primer, Gribskov, M. and Devereux, J., eds., M Stockton Press, New York, 1991.
27. Karlin et al. Proc. Natl. Acad. Sci. USA. 1993. 90:5873-5877.
28. Needleman et al. J. Mol. Biol. 1970. 48:444-453).
29. Ausubel F. M. et al., eds., 1989, Current Protocols in Molecular Biology, Vol. I, Green Publishing Associates, Inc., and John Wiley & Sons, Inc., New York, at p. 2.10.3.
30. Pfeifer A., Verma I. M. Gene therapy: promises and problems. Annu Rev Genomics Hum Genet. 2001. 2:177-211.
31. Amado R. G., Chen I. S. Lentiviral Vectors—the promise of gene therapy within reach? Science. 1999. 285:674-676.
32. Trono D. (2002) Lentiviral Vectors. Springer-Verlag: New York/Berlin/Heidelberg.
33. Chater T E, Henley J M, Brown J T, Randall A D. Voltage- and temperature-dependent gating of heterologously expressed channelrhodopsin-2. J Neurosci Methods. 2010. 193:7-13.
34. Nagel G, Brauner M, Liewald J F, Adeishvili N, Bamberg E, Gottschalk A. Light activation of channelrhodopsin-2 in excitable cells of caenorhabditis elegans triggers rapid behavioral responses. Curr Biol. 2005. 15:2279-2284.
35. Bruegmann T, Malan D, Hesse M, Beiert T, Fuegemann C J, Fleischmann B K, Sasse P. Optogenetic control of heart muscle in vitro and in vivo. Nat Methods. 2010. 7:897-900.
36. Gunaydin L A, Yizhar O, Berndt A, Sohal V S, Deisseroth K, Hegemann P. Ultrafast optogenetic control. Nat Neurosci. 2010; 13:387-392.
37. Bien, B. H., L. Yin, and E. Entcheva, Calcium instabilities in mammalian cardiomyocyte networks. Biophys J. 2006. 90(7):2628-40.
38. Yu H, Gao J, Wang H, Wymore R, Steinberg S, McKinnon D, Rosen M R, Cohen I S. Effects of the renin-angiotensin system on the current i(to) in epicardial and endocardial ventricular myocytes from the canine heart. Circ. Res. 2000. 86:1062-1068.
39. Valiunas V, Kanaporis G, Valiuniene L, Gordon C, Wang H, Li L, Robinson R B, Rosen M R, Cohen I S; Brink P R. Coupling an hcn2 expressing cell to a myocyte creates a two cell pacing unit. J Physiol. 2009. 587:5111-26.
40. Valiunas V, Beyer E C, Brink P R. Cardiac gap junction channels show quantitative differences in selectivity. Circ. Res. 2002. 91:104-111.
41. Entcheva, E. and H. Bien, Macroscopic optical mapping of excitation in cardiac cell networks with ultra-high spatiotemporal resolution. Progress in Biophysics and Molecular Biology, 2006. 92(2):232-257.
42. Entcheva E. and H. Bien, Tension development and nuclear eccentricity in topographically controlled cardiac syncytium. J Biomed Microdev. 2003. 5:163-168.
43. Malmivuo J, Plonsey R. Bioelectromagnetism. New York: Oxford University Press, Ch. 3 (http://www.bem.fi/book/index.htm); 1995.
44. Boyden E S, Zhang F, Bamberg E, Nagel G, Deisseroth K. Millisecond-timescale, genetically targeted optical control of neural activity. Nat Neurosci. 2005. 8:1263-1268.
45. Wang H, Peca J, Matsuzaki M, Matsuzaki K, Noguchi J, Qiu L, Wang D, Zhang F, Boyden E, Deisseroth K, Kasai H, Hall W C, Feng G, Augustine G J. High-speed mapping of synaptic connectivity using photostimulation in channelrhodopsin-2 transgenic mice. Proc Natl Acad Sci USA. 2007. 104:8143-8148.

1. An optically-controlled biological device comprising:
(a) a non-excitable cell expressing a light-gated ion channel protein and capable of forming a gap junction channel with a target cell when implanted in a subject, and
(b) an optical stimulation unit.

2. The device of claim 1, wherein the target cell is a cardiomyocyte.

3. The device of claim 1, wherein the device is a cardiac pacemaker.

4. The device of claim 1, wherein the device is an implantable cardioverter defibrillator.

5. The device of claim 1, wherein the device is an optical-mechanical actuator.

6. The device of claim 1, wherein the light-gated ion channel protein is selected from the group consisting of channelrhodopsin-1 (ChR1), channelrhodopsin-2 (ChR2), Volvox channelrhodopsin (VChR1), halorhodopsin (Halo/NpHR), archaerhodopsin-3 (Arch), Leptosphaeria maculans rhodopsin (Mac), and combinations thereof.

7. The device of claim 6, wherein the light-gated ion channel protein is channelrhodopsin-2 (ChR2).

8. The device of claim 1, wherein the non-excitable cell is engineered to express one or more connexin proteins.

9. The device of claim 8, wherein the non-excitable cell is engineered to express a connexin selected from the group consisting of connexin 40, connexin 43, connexin 45, and combinations thereof.

10. The device of claim 1, wherein the non-excitable cell is selected from the group consisting of a stem cell, an endothelial cell, a fibroblast cell, and an adipocyte.

11. The device of claim 1, wherein the non-excitable cell is a human mesenchymal stem cell.

12. The device of claim 1, wherein the devices comprises more than about 100,000 of the non-excitable cells.

13. The device of claim 1, wherein the ratio of non-excitable cells to target cells is from about 1:20 to about 1:100 when the non-excitable cells are delivered to the subject.

14. The device of claim 1, wherein the optical stimulation unit comprises a light source wherein the light source is selected from the group consisting of a laser, a laser diode, a light emitting diode, an organic light emitting diode, an incandescent light source, and combinations thereof.

15. The device of claim 1, wherein the optical stimulation unit comprises one or more sensors for detecting cardiac pacing in the atrium, ventricles, or both.

16. A method of treating a subject with a cardiac pacing disorder, comprising:
(a) delivering in proximity to the heart of the subject a non-excitable cell expressing a light-gated ion channel protein; wherein the non-excitable cell forms a gap junction channel with a cardiomyocyte of the subject; and
(b) providing an optical stimulation unit.

17. The method of claim 16, wherein the cardiac pacing disorder is selected from the group consisting of cardiac arrhythmia, reentrant arrhythmia, bradycardia, tachycardia, sinus bradycardia, sinus tachycardia, ventricular tachycardia, supraventricular tachycardia, ventricular fibrillation, atrial flutter, atrial fibrillation, pro-arrhythmic cardiac alternans, sinus node dysfunction, first degree heart block, type 1 second degree heart block, type 2 second degree heart block, third degree heart block, SA block, and SV block.

18. The method of claim 16, wherein the light-gated ion channel protein is selected from the group consisting of channelrhodopsin-1 (ChR1), channelrhodopsin-2 (ChR2), Volvox channelrhodopsin (VChR1), halorhodopsin (Halo/NpHR), archaerhodopsin-3 (Arch), Leptosphaeria maculans rhodopsin (Mac), and combinations thereof.

19. The method of claim 15, wherein the light-gated ion channel protein is channelrhodopsin-1 (ChR1).

20. The method of claim 16, wherein the non-excitable cell is engineered to express one or more connexin proteins.

21. The method of claim 16, wherein the non-excitable cell is engineered to express a connexin selected from the group consisting of connexin 40, connexin 43, connexin 45, and combinations thereof.

22. The method of claim 16, wherein the non-excitable cell is selected from the group consisting of a stem cell, an endothelial cell, a fibroblast, or an adipocyte.

23. The method of claim 22, wherein the non-excitable cell is a human mesenchymal stem cell.

24. The method of claim 16, wherein the method comprises delivering more than about 100,000 of the non-excitable cells.

25. The method of claim 16, wherein the method comprises delivering non-excitable cells such that the ratio of non-excitable cells to cardiomyocytes is from about 1:20 to about 1:100.

26. The method of claim 16, wherein the non-excitable cell is delivered to one or more of the location selected from the group consisting of the sinoatrial node, the atrioventricular node, Bachmann's bundle, the atrioventricular junction region, His branch, left bundle branch, right bundle branch, Purkinje fibers, right atrial muscle, left atrial muscle, and ventricular muscle.

27. The method of claim 16, wherein the optical stimulation unit comprises a light source wherein the light source is selected from the group consisting of a laser, a laser diode, a light emitting diode, an organic light emitting diode, an incandescent light source and combinations thereof.

28. The method of claim 16, wherein the optical stimulation unit comprises one or more sensors for detecting cardiac pacing in the atrium, ventricles, or both.
